(19) United States
(12) Patent Application Publication
(10) Pub. No.: US 2026/0365980 A1
(43) Pub. Date: Nov. 19, 2026

(54) METHOD FOR PRODUCING DENDRITIC CELL FORMULATION INCLUDING DENDRITIC CELLS THAT STIMULATE NKT CELLS

(71) Applicant: DC-BIOTECH Ltd., Kyoto-shi, Kyoto (JP)

(72) Inventors: Shigeru KAGATA, Kyoto (JP); Yusuke TORIKAI, Kyoto (JP); Yuki ISHIHARA, Kyoto (JP); Yasuyuki ISHII, Tokyo (JP); Risa NOZAWA, Tokyo (JP)

(73) Assignee: DC-BIOTECH Ltd., Kyoto-shi, Kyoto (JP)

(21) Appl. No.: 19/121,944

(22) PCT Filed: Oct. 13, 2023
(86) PCT No.: PCT/JP2023/037210

§ 371 Date: Apr. 17, 2025

(30) Foreign Application Priority Data
Oct. 17, 2022 (JP) 2022-166035

(51) Int. Cl. C12N 5/0784; A61K 35/15

(52) U.S. Cl. CPC C12N 5/0639; A61K 35/15; C12N 2500/36; C12N 2501/22; C12N 2501/2304

(57) ABSTRACT

Method for producing a dendritic cell preparation which comprises an adhesion step comprising placing collected monocytes into a culture vessel with a liquid medium and statically incubating them to allow some of said monocytes to adhere to the inner surface of the vessel, a non-adherent cell removal step comprising removing non-adherent cells, a differentiation step comprising adding predetermined factors to the culture vessel to allow the monocytes adhered to the inner surface of the culture vessel to differentiate into immature dendritic cells, a pulsing step comprising pulsing α-Galactosylceramide into the culture vessel in which said immature dendritic cells are present in a non-adhering state, and an NKT-stimulating dendritic cell induction step comprising inducing NKT-stimulating dendritic cells which are dendritic cells that stimulate NKT cells, from the immature dendritic cells, wherein the preparation is for reintroducing into the body of the beneficiary from whom the blood has been collected.

### TECHNICAL FIELD

The present invention relates to a method for producing a dendritic cell preparation comprising dendritic cells that stimulate NKT cells (wherein “T” stands for thymus) with fewer side effects, for one or more of treating cancer, preventing cancer, or enhancing immunity in a healthy person. In particular, the invention relates to a method for producing a dendritic cell preparation derived from autologous cells collected from the beneficiary, and to blood to which the dendritic cell preparation produced by said method has been added.

### BACKGROUND ART

In recent years, cancer immunotherapy or methods for enhancing immunity in healthy individuals, both targeting NKT cells, have been developed. These therapies do not directly target cancer cells or other cells themselves, but instead activate NKT cells, which serve as the command center of the immune system. The adjuvant effect of the activated NKT cells (the effect of enhancing the overall immune response by activating certain immune cells, which in turn induce the activation of other immune cells) leads to a strong anti-tumor effect. Furthermore, since this is not restricted to targeting specific antigens, it is expected to be effective not only against newly mutated cancer cells but also against various non-cancerous pathogens, including bacteria, viruses, and parasites.

Patent Document 1 describes a method in which mononuclear cells are placed into a first culture vessel and allowed to stand so that some of the mononuclear cells attach to the bottom surface of the vessel. The non-adherent cells other than the cells adhered to the bottom surface of the vessel, are collected and stored. Monocytes among the remaining cells attached to the bottom surface, are subjected to a predetermined treatment to cause differentiation into immature dendritic cells, followed by additional predetermined treatment to induce maturation. Thereafter, α-Galactosylceramide (hereinafter referred to as α-Galactosylceramide) is added to the mature dendritic cells to induce NKT cell-stimulating dendritic cells from the mature dendritic cells. The induced NKT cell-stimulating dendritic cells are then placed into a second culture vessel together with the previously stored non-adherent cells for co-culture, thereby inducing NKT cells from some of the non-adherent cells.

### Prior Art Document

### Patent Documents

Patent Document 1: JP6854290B

### SUMMARY OF THE INVENTION

### Problems to be Solved by the Invention

Patent Document 1 describes that it is known that NKT cells produce a large amount of IFN-γ (interferon-gamma) when activated and that when CD1d molecules are presented on the surface of antigen-presenting cells (dendritic cells), an activation signal is transmitted via the T-cell receptor on the surface of NKT cells, resulting in the activation of NKT cells.

Regarding the cell composition containing the dendritic cells capable of stimulating NKT cells and NKT cells disclosed in Patent Document 1, the proportion of NKT cells is indicated. However, since the amount of IFN-γ production, which plays a role in inducing various effects on cancer cells, is not disclosed, the effectiveness is not explicitly demonstrated. Additionally, the induction for stimulating NKT cells is carried out after the dendritic cells have undergone maturation. However, there is no description discussing the temporal relationship between the timing of induction and the timing of maturation.

In order to solve these problems, the present inventors have conducted research and development and have developed the preparation of the present invention that produces a greater amount of IFN-γ and initiates IFN-γ production earlier than the method disclosed in Patent Document 1. The present invention enables an increased amount of IFN-γ production by NKT cells compared to the prior art (Patent Document 1) as a result of optimizing the timing of performing induction for stimulating NKT cells on dendritic cells. The produced IFN-γ stimulates and activates both pathways of the innate and acquired immune systems in the human body. As a result, the greater effects from both immune pathways stimulated by the large amount of IFN-γ produced can be expected to contribute to cancer treatment and other applications. Accordingly, the present invention provides a method of producing a dendritic cell preparation comprising NKT cell-stimulating dendritic cells, which is expected to achieve higher therapeutic efficacy.

### Means to Solve the Problems

First, the means for solving the problem is summarized. The timing for pulsing the ligand to dendritic cells is more effective when the pulsing is performed at the stage where the dendritic cells are immature. Moreover, this does not mean that pulsing can be performed at any arbitrary timing within the immature stage; rather, greater effects can be achieved by pulsing at an appropriate timing within the immature stage. The specific means for solving the problem are described below.

In order to solve the above-mentioned problems related to dendritic cell preparations, the present application provides, as a first invention,
a method for producing a dendritic cell preparation comprising dendritic cells that stimulate natural killer T (NKT) cells for one or more of the following purposes: treating cancer, preventing cancer, or enhancing immunity which comprises
(1) an adhesion step comprising placing monocytes collected from a beneficiary (monocytes or peripheral blood containing monocytes collected by blood apheresis or blood collection) into a culture vessel with a liquid medium and statically incubating them to allow some of said monocytes to adhere to the inner surface of the vessel,
(2) a non-adherent cell removal step comprising removing non-adherent cells including monocytes, which are cells other than those adhered to the inner surface of the culture vessel,
(3) a differentiation step comprising adding predetermined factors to the culture vessel to allow the monocytes adhered to the inner surface of the culture vessel to differentiate into immature dendritic cells,
(4) a pulsing step comprising pulsing α-Galactosylceramide into the culture vessel in which said immature dendritic cells are present in a dominant state, and
(5) an NKT-stimulating dendritic cell induction step comprising inducing NKT-stimulating dendritic cells from the immature dendritic cells to NKT-stimulated dendritic cells, which are dendritic cells that stimulate NKT cells,
wherein the dendritic cell preparation is a NKT cell-stimulating dendritic cell preparation for reintroducing into the body of the beneficiary from whom the monocytes were collected, to stimulate NKT cells.

Furthermore, as a second invention, the present application provides a method for producing a dendritic cell preparation, based on the first invention, wherein the pulsing step (4) is carried out on day 3 or day 4, wherein day 0 is defined as the day on which the predetermined factors are added in the differentiation step (3).

Furthermore, as a third invention, the present application provides a method for producing a dendritic cell preparation, based on the first invention, wherein the pulsing step (4) is carried out within a period of 60 to 108 hours after the predetermined factors have been added in the differentiation step (3).

Furthermore, as a fourth invention, the present application provides a method for producing a dendritic cell preparation according to claim 1, based on the first invention, wherein the pulsing step (4) is carried out by day 4 or day 5, wherein day 0 is defined as the day on which the monocytes collected from the beneficiary are placed into the culture vessel in the adhesion step (1).

Furthermore, as a fifth invention, the present application provides a method for producing a dendritic cell preparation, based on the first invention, wherein the pulsing step (4) is carried out within a period of 84 to 132 hours after the monocytes collected from the beneficiary have been placed into the culture vessel in the adhesion step (1).

Furthermore, as a sixth invention, the present application provides a method for producing a dendritic cell preparation, based on the first invention, wherein the pulsing step (4) is carried out on day 4 or day 5, wherein day 0 is defined as the day on which the non-adherent cells including monocytes, which are cells other than those adhered to the inner surface of the culture vessel, are removed in the non-adherent cell removal step (2).

Furthermore, as a seventh invention, the present application provides a method for producing a dendritic cell preparation, based on the first invention, wherein the pulsing step (4) is carried out within a period of 84 to 132 hours after the non-adherent cells including monocytes, which are cells other than those adhered to the inner surface of the culture vessel, are removed in the non-adherent cell removal step (2).

Furthermore, as an eighth invention, the present application provides a method for producing a dendritic cell preparation, based on any one of the first invention to the seventh invention, wherein the concentration of α-Galactosylceramide to be pulsed in the pulsing step (4) is from 300 to 1000 ng/mL.

Furthermore, as a ninth invention, the present application provides a blood from a beneficiary, to which the dendritic cell preparation produced by the production method according to any one of the first invention to the seventh invention, has been added.

Furthermore, as a tenth invention, the present application provides a blood from a beneficiary, to which the dendritic cell preparation produced by the production method according to the eighth invention, has been added.

### Effects of the Invention

According to the present method for producing a dendritic cell preparation that stimulates NKT cells, for reintroducing into the body of a beneficiary from whom the monocytes were collected, the amount of IFN-γ production by NKT cells can be greater than that in the prior art (Patent Document 1). The produced IFN-γ stimulates and activates both pathways of the innate and acquired immune systems in the human body. As a result, the greater effects from both immune pathways stimulated by the large amount of IFN-γ can be expected to contribute to cancer treatment or the like, thereby providing a method for producing a dendritic cell preparation comprising NKT cell-stimulating dendritic cells, that is expected to achieve higher therapeutic efficacy.

### BRIEF DESCRIPTION OF THE DRAWINGS

FIG. 1 Graph showing the relationship between the pulsing timing of α-Galactosylceramide during the production of the dendritic cell preparation of Embodiment 2 and the NKT cell count ratio after co-culturing.

FIG. 2a Graph 1 showing the relationship between the pulsing timing of α-Galactosylceramide during the production of the dendritic cell preparation of Embodiment 2 and the maximum NKT cell count ratio after co-culturing under various conditions (when the day of adding the predetermined differentiation factor is set as day 0).

FIG. 2b Graph 2 showing the relationship between the pulsing timing of α-Galactosylceramide during the production of the dendritic cell preparation of Embodiment 2 and the maximum NKT cell count ratio after co-culturing under various conditions (when the start of culturing is set as the starting point).

FIG. 3 Graph showing the relationship between the pulsing timing of α-Galactosylceramide during the production of the dendritic cell preparation of Embodiment 3 and the NKT cell count ratio after co-culturing.

FIG. 4a Graph 1 showing the relationship between the pulsing timing of α-Galactosylceramide during the production of the dendritic cell preparation of Embodiment 3 and the maximum NKT cell count ratio after co-culturing under various conditions (when the day of adding the predetermined differentiation factor is set as day 0).

FIG. 4b Graph 2 showing the relationship between the pulsing timing of α-Galactosylceramide during the production of the dendritic cell preparation of Embodiment 3 and the maximum NKT cell count ratio after co-culturing under various conditions (when the start of culture is set as the starting point).

FIG. 5 Graph showing the relationship between the concentration of α-Galactosyleeramide to be pulsed during the production of the dendritic cell preparation of Embodiment 4 and the amount of IFN-γ produced after co-culturing.

FIG. 6 NK cell activity values and IFN-γ production levels when the dendritic cell preparation of the present invention is used in the human body.

FIG. 7 Comparative diagram of the effects of the invention (the amount of IFN-γ produced after co-culturing) between the prior art (Patent Document 1) and the present invention.

### DESCRIPTION OF EMBODIMENTS

First, prior to the description of the embodiments of the present invention, an explanation of the body's immunity will be provided.

Hereinafter, in this specification, the effects of the preparation of the present invention on cancer cells will be described as an example; however, similar effects can also be obtained against other pathogens or the like. The immunity can be classified into innate immunity, which organisms inherently possess, and acquired immunity, which organisms acquire over time.

Innate immunity is an immunity inherently present in the human body from birth and is primarily composed of phagocytes, which eat and eliminate foreign substances such as bacteria and viruses. Phagocytes possess receptors that recognize molecules and structures and can be broadly classified into those that process and eliminate foreign substances and those that play a role in transmitting signals between cells. Examples of innate immune cells include eosinophils, neutrophils, basophils, macrophages, dendritic cells, and natural killer (NK) cells.

On the other hand, acquired immunity is an immunity that memorizes pathogens in the body and enables their effective elimination when they are encountered again. Compared to innate immunity, acquired immunity requires a longer response time; however, it possesses diversity that allows it to respond to a wide range of pathogens. Acquired immune cells include D cells and T cells, and T cells further include helper T cells, killer T cells, and regulatory T cells. B cells change into plasma cells or memory B cells depending on the physiological state.

In addition, there are NKT cells that activate both innate and acquired immunity.

Among the immune cells mentioned above, NKT cells are directly activated by the present dendritic cell preparation, but NK cells, dendritic cells, T cells (helper T cells and killer T cells), B cells or the like are also activated by the adjuvant effect of IFN-γ produced through the activation of NKT cells.

The following is a brief description of immune cells and substances related to immune function that are involved in the action of the dendritic cell preparation produced by the present method for producing a dendritic cell preparation.

### Explanation of Terms for Immune Cells and Substances Related to Immune Function

### Term Explanation: Antigen

Antigen is a foreign substance that enters the body and serves as a marker for the acquired immune cells to recognize it as a target of attack. In the case of a cancer antigen, immune cells recognize it as a target for attack based on protein structures that are not present in normal cells.

### Term Explanation: Antibody

An antibody is a general term for proteins called immunoglobulins. Cancer antibodies bind to cancer antigens on cancer cells and attack the cancer cells through the following three mechanisms:

Neutralization: Inhibits the proliferation of cancer cells.

ADCC activity: Functions as a marker for killer T cells and NK cells, aiding in attacking cancers.

CDC activity: An Action in which complements (a type of protein synthesized in the body and present in serum, a substance involved in immune responses such as infection defense, including nine types from C1 to C9) bind to antibodies that have bound to cancer antigens on cancer cells, triggering the sequential activation of complements from C1 to C9, leading to the formation of pores in the cancer cell membrane, thereby damaging and killing the cancer cells.

### Term Explanation: Cytokine

Cytokine is a general term for proteins secreted by immune cells, bioactive substances involved in intercellular interactions. Cytokines transmit signals to target cells and induce various cellular responses such as cell activation, cell proliferation, differentiation, cell death, and functional expression. Interleukins, interferons, and tumor necrosis factors are examples of cytokines.

### Term Explanation: Cytokine: Interleukin

Interleukin, a type of cytokine, has been identified in more than 30 different types. It is particularly secreted in large amounts by lymphocytes, which are a type of white blood cell that fights against foreign substances in the body, as well as by phagocytes such as macrophages and neutrophils. Interleukins are designated by numerical names, such as interleukin-2 (hereinafter, interleukin is abbreviated as IL; for example, interleukin-4 is abbreviated as IL-4). The following describes some interleukins, their sources and functions.

### Term Explanation: Cytokine: Interleukin: IL-4

IL-4 is one of the Th2 cytokines and is produced by activated type 2 helper T cells (Th2 cells), NKT cells or the like. It promotes the proliferation and differentiation of antigen-presenting cells and affects B cells, T cells, macrophages, monocytes and the like. Naïve T cells differentiate into Th2 cells upon stimulation by cytokines such as IL-4 and IL-13. When IL-4 acts on activated B cells, a class switch occurs in which the produced immunoglobulin (antibody) changes from IgM to IgE and IgG1. Thus, IL-4 is involved in the humoral immune system.

NKT cells are activated when they recognize α-Galactosylceramide presented by antigen-presenting cells (dendritic cells), and produce IL-4 and IFN-γ.

### Term Explanation: Cytokine: Interleukin: IL-12

IL-12 is produced by phagocytes (cells responsible for cellular immunity that migrate between tissues and exhibit phagocytic activity in an animal body: macrophage, monocyte, neutrophil or the like) and dendritic cells. IL-12 is a NK cell-stimulating factor and, together with IFN-γ, acts on naive T cells to induce their differentiation into Th1 cells, a subset of helper T cells. Th1 cells produce Th1 cytokines such as IFN-γ, which are involved in cellular immunity by activating macrophages and killer T cells.

α-Galactosylceramide induces dendritic cells (antigen-presenting cells) to produce IL-12. IL-12 produced by antigen-presenting cells acts on NKT cells to produce IFN-γ or exert cytotoxic activity on target cells expressing Fas antigen (Fas receptor) via Fas ligand expressed on the surface of NKT cells.

### Term Explanation: Cytokine: Interferon

Interferon, a type of cytokine, has a function to activate natural killer (NK) cells, which attack cancer cells and virus-infected cells, as well as macrophages to promote their function to suppress proliferation of viruses and tumor cells and to phagocytize cancer cells(adjuvant effect). Interferon has been approved by the government as an antiviral and anticancer drug, and is used in the treatment of multiple myeloma, brain tumors, and renal cancer.

### Term Explanation: Cytokine: Interferon: IFN-γ

IFN-γ is a cytokine essential for both the innate immune system and the acquired immune system. IFN-γ is primarily produced by NK cells and NKT cells as a part of the innate immune response. After the induction of antigen-specific immunity, it is produced by Th1 cells, killer T cells, and macrophages. IFN-γ accelerates the processing of a large amount of invading non-self-antigens and enhances cellular immune responses, It is a key activator of macrophages, stimulating them to phagocytize and kill bacteria. Additionally, IFN-γ promotes NK cell activity and induces naive T cells to differentiate into Th1 cells.

### Term Explanation: Cytokine: Tumor Necrosis Factor

Tumor Necrosis Factor (TNF) is a substance secreted by lymphocytes and other cells that induces necrosis of tumor cells. TNF acts on almost all malignant cells and also promotes immune responses. In a narrow sense, TNF includes three types: TNF-α, TNF-β (lymphotoxin (LT)-α), and LT-β.

### Term Explanation: Cytokine: Tumor Necrosis Factor: TNF-α

TNF-α is primarily produced by macrophages and was discovered as a cytokine that induces hemorrhagic necrosis in solid tumors. Generally, the term tumor necrosis factor is often used to refer to TNF-α. TNF-α is involved in the expression of cell adhesion molecules and the induction of apoptosis. Apoptosis refers to a regulated and controlled cell suicide, i.e., programmed cell death.

### Term Explanation: NK Cell

NK cells possess various receptors that catch abnormal cells. When NK cells detect cells that differ from normal cells constituting human body, such as virus-infected cells or cancer cells (as NK cells retain a memory of normal cells constituting human body), they recognize these cells as abnormal and exert cytotoxic effects on the abnormal cells.

### Term Explanation: Dendritic Cell

Dendritic cells are antigen-presenting cells. They acquire the ability to present antigens specific to foreign substances (e.g., pathogen- or virus-infected cells or cancer cells) through uptake (phagocytosis) of the foreign substances or acquire the ability to present antigens by receiving antigens presented by macrophages or the like. Dendritic cells play a role in presenting these antigens to T cells, enabling T cells to recognize them as targets for attack. Dendritic cells possess phagocytic activity to phagocytize viruses and cancer cells, and through this process they acquire antigens and develop the ability to present said antigens to other immune cells. Early dendritic cells (immature dendritic cells) have the ability to take up antigens (phagocytic activity) but cannot activate naive T cells (which can be said to have no antigen-presenting ability). Upon maturation, mature dendritic cells acquire antigen-presenting ability that enables them to activate T cells, but they lose their ability to take up antigens (phagocytosis).

### Term Explanation: Helper T Cell

When presented with antigens by dendritic cells or the like, helper T cells recognize them as targets of attack and instruct killer T cells to attack. They also produce cytokines to activate B cells. Furthermore, when presented with antigens by B cells, they determine whether the antigens originate from a harmful foreign substance and transmit the results to B cells. In addition, they also assist with antibody production.

### Term Explanation: Killer T Cells

Killer T cells recognize antigens presented by MHC class I molecules (MHC class I molecules bind peptides resulting from the degradation of cytoplasmic proteins, primarily by the proteasome (proteolytic enzyme complex) (as a marker for indicating themselves as humans). The MHCI peptide complex is then inserted through the endoplasmic reticulum membrane to the outside of the cell membrane. The epitope peptide is bound to the extracellular portion of the MHC I-peptide molecule. In this way, the MHC I-peptide molecule presents intracellular proteins to killer T cells. However, in some cases, MHC I-peptide molecules present peptides derived from foreign proteins, a process known as cross-presentation.) distinguish between self and non-self and exert cytotoxic effects on non-self-cells. Thus, the T cell response, which is acquired immunity, begins with antigen presentation of antigen peptides by MHC molecules.

Under the direction of helper T cells, killer T cells kill and eliminate cells that are deemed harmful or unnecessary to the organism, such as virus-infected cells and cancer cells.

### Term Explanation: B Cells

B cells are stimulated by cytokines produced by helper T cells and produce antibodies. Even after the antigen has been eliminated, they serve as memory B cells to retain a memory of the antigen and prepare for the next invasion.

### Term Explanation: NKT Cells

NKT cells are a cell that possesses characteristics of both T cells and NK cells. Upon activation, they produce cytokines (IFN-γ) and play a role in inducing immunostimulatory or suppressive therapy responses. The most expected effect of the present preparation is the adjuvant action, which activates various immune cells in the body and, this is an important point, also promotes their proliferation.

### Effects of the Dendritic Cell Preparation That Stimulates NKT Cells

The primary expected effects of NKT cell activation by the present preparation include both nonspecific and specific immune responses, such as promotion of dendritic cell maturation, adjuvant effects (activation and proliferation of various immune cells), induction of apoptosis in cancer cells, and inhibition of angiogenesis. Apoptosis refers to a regulated and controlled cell suicide, i.e., programmed cell death.

As described above, by using the NKT cell-stimulating dendritic cell preparation, which activates the innate immune system, various immune functions in the body can be activated, enabling the attack on cancer cells.

The detailed embodiments will be described below. The substances used during the production method are common to all embodiments. The manufacturer and other relevant information will be provided only at their first mention.

### <Embodiment 1: Overview> Primarily Corresponding to Claim 1

Provided is a production method comprising transforming monocytes collected from a beneficiary (monocytes or peripheral blood containing monocytes collected by blood apheresis or blood collection) into dendritic cells that stimulate NKT cells to give a dendritic cell preparation.

As used herein, the term “beneficiary” refers to an individual who wishes to benefit from the effects of the administration of the dendritic cell preparation for one or more of purposes, including treating cancer, preventing cancer, or enhancing immunity, and who provides their own monocytes as the raw material for said preparation.

### Embodiment 1: Production Method

First, an overview of the method for producing the NKT cell-stimulating dendritic cell preparation of the present invention will be described. Next, the effects of the present preparation will be explained below.

### Embodiment 1: (1) Adhesion Step: Preparation of Monocytes

Monocytes collected from a beneficiary (monocytes or peripheral blood containing monocytes collected by blood apheresis or blood collection) are prepared. The term “preparation” refers to bringing the monocytes collected from a beneficiary (monocytes or peripheral blood containing monocytes collected by blood apheresis or blood collection) to the manufacturing site and does not include the blood collection process itself. In this adhesion step, preparation may also include bringing a culture vessel or a liquid culture media for later use to the manufacturing site and making them ready for use. Unless otherwise specified herein, the term “culture vessel” refers to a vessel for culturing that contains a medium, such as a liquid culture medium.

The term “apheresis” refers to a process in which blood is separated from the body, desired blood components or factors are separated from the blood using a specialized equipment, and the remaining is returned to the body. In the present invention, approximately 100 cc of blood components are collected by separating peripheral blood mononuclear cells including monocytes as blood components. The blood components remaining after the separation of peripheral blood mononuclear cells including monocytes contain plasma and platelets other than the desired blood components described above. Apheresis is very stressful for the body because an anticoagulant is mixed in the blood before separation of the peripheral blood mononuclear cells including monocytes to prevent the blood from coagulating, and then the anticoagulant is returned to the body, and the apheresis for separation of the peripheral blood mononuclear cell including monocytes takes a long time.

It is also acceptable to collect 100 to 400 mL of peripheral blood instead of collecting the sample through apheresis. While apheresis yields a higher proportion of monocytes, peripheral blood collection may be preferable as it can be performed in a shorter time without the use of anticoagulants.

### Embodiment 1: (1) Adhesion Step: Static Incubation

Next, mononuclear cells are collected from the collected peripheral blood mononuclear cells containing monocytes by a density gradient centrifugation with Ficoll (registered trademark). (Ficoll (registered trademark) is a component of Ficoll-Paque (registered trademark), which is used to separate blood into its components (such as red blood cells, white blood cells or the like.) The collected mononuclear cells are then placed into a culture vessel containing a liquid culture medium. It is preferable to place the mononuclear cells collected from the beneficiary into a culture vessel containing a medium (preferably a liquid culture medium) within 72 hours after collection. The time point at which mononuclear cells containing monocytes are placed into the culture vessel containing a medium (preferably a liquid culture medium) is defined as the start of culture. Unless otherwise specified herein, the phrase “placing monocytes into a culture vessel” shall be understood to mean placing them into a culture vessel containing a medium (preferably a liquid culture medium). The culture vessel is then statically incubated for approximately 30 minutes under a sterile 37 °C thermostatic environment. Unless otherwise specified, subsequent steps following static incubation are also performed under a sterile 37 °C thermostatic environment. The static incubation period may be longer than 30 minutes. If it is shorter than 30 minutes, an insufficient number of monocytes will adhere to the inner surface of the vessel. Therefore, it is preferable to statically incubate for at least approximately 30 minutes.

As used herein, the term “adhesion” to the inner surface of a culture vessel does not mean intentionally pressing cells against the inner surface of the culture vessel to bring them into contact, nor does it mean using any substance as an adhesive to affix the cells in a manner that prevents them from easily detaching from the inner surface of the culture vessel. Rather, “adhesion” refers to maintaining a state in which the cells remain attached to the inner surface without the application of any external force other than gravity after coming into contact with the inner surface.

### Embodiment 1: (2) Non-Adherent Cell Removal Step: Removal of Non-Adherent Cells

After the above-mentioned static incubation (e.g., after static incubation for 30 minutes), non-adherent cells, including monocytes, that remain suspended in the liquid culture medium without adhering to the inner surface of the culture vessel, are removed (transferred to another vessel). Cells that have adhered to the inner surface of the vessel (monocyte fraction) remain in the culture vessel containing the liquid medium.

The removed non-adherent cells, including monocytes, are preferably either separately cultured or cryopreserved. The removed non-adherent cells (including NKT cells) can be used for efficacy verification of the produced preparation by co-culturing them with said preparation. After the removal of non-adherent cells, a detachment step comprising detaching the adherent cells (monocyte fraction) attached to the inner surface of the vessel and suspending them in a liquid medium may be provided. Since monocytes primarily adhere to the inner surface of the vessel during the above-described static incubation, monocytes will mainly remain suspended in the liquid culture medium after detachment.

### Embodiment 1: (3) Differentiation Step: Differentiation of Monocytes Into Immature Dendritic Cells

To the culture vessel containing the liquid culture medium from which non-adherent cells have been removed, granulocyte-macrophage colony-stimulating factor (hereafter, GM-CSF) at 50 ng/ml (approximately 30 ng/ml to 70 ng/ml is preferable. If the concentration is lower, the stimulation is too weak, resulting in slower differentiation. Conversely, if the concentration is higher, the differentiation rate reaches a plateau, making it economically inefficient.) and interleukin-4 (hereinafter, IL-4) at 50 ng/ml (approximately 30 ng/ml to 70 ng/ml is preferable. If the concentration is lower, the stimulation is too weak, resulting in slower differentiation. Conversely, if the concentration is higher, the differentiation rate reaches a plateau, making it economically inefficient.) are added as predetermined factors for inducing the differentiation of monocytes into immature dendritic cells. After the addition, the culture is continued. By adding GM-CSF and IL-4 to stimulate the monocytes, the monocytes in the culture differentiate into immature dendritic cells. The substances used for differentiation stimulation are preferably GM-CSF and IL-4, but are not limited thereto. The addition of the stimulating substances causes the presence of a very large number (presence in a dominant state) of immature dendritic cells over undifferentiated monocytes and mature dendritic cells in the culture vessel. The differentiated immature dendritic cells constitute the majority of the cells in the culture vessel, preferably 80% or more, and more preferably 90% or more.

### Embodiment 1: (4) Pulsing Step: Pulsing of α-Galactosylceramide

As described above, α-Galactosylceramide is pulsed into the liquid culture medium in the culture vessel, where the immature dendritic cells account for more than half of the total cell population. The α-Galactosylceramide used was KRN7000, a code name by Kyowa Kirin Co., Ltd. By being pulsed with α-Galactosylceramide, the immature dendritic cells become immature dendritic cells capable of stimulating NKT cells after maturation. α-Galactosylceramide is a glycolipid antigen essential for activating NKT cells. The unique feature of the present invention is that α-Galactosylceramide is given directly to immature dendritic cells. Generally, mature dendritic cells acquire the antigen-presenting ability for α-Galactosylceramide through phagocytosis of various foreign substances, but in the present invention, α-Galactosylceramide is given (pulsed) to dendritic cells in the immature stage to confer the antigen-presenting ability on the dendritic cells after maturation. Although a prior art, Patent Document 1, describes pulsing mature dendritic cells with α-Galactosylceramide gives antigen-presenting ability, the inventors of the present application have confirmed that pulsing immature dendritic cells with α-Galactosylceramide is more effective in presenting antigens to NKT cells and activating NKT cells.

### Embodiment 1: (5) NKT-Stimulating Dendritic Cell Induction Step: Maturation of Dendritic Cells

After pulsing with α-Galactosylceramide as described above, GM-CSF (5 ng/ml: approximately 3 ng/ml to 7 ng/ml is preferable. If the concentration is lower, the stimulation is too weak, resulting in slower differentiation. Conversely, if the concentration is higher, the differentiation rate reaches a plateau, making it economically inefficient.), IL-4 (5 ng/ml: approximately 3 ng/ml to 7 ng/ml is preferable. If the concentration is lower, the stimulation is too weak, resulting in slower differentiation. Conversely, if the concentration is higher, the differentiation rate reaches a plateau, making it economically inefficient.), OK-432 (10 ng/ml: approximately 7 ng/ml to 13 ng/mL is preferable. If the concentration is lower, the stimulation is too weak, resulting in slower differentiation. Conversely, if the concentration is higher, the differentiation rate reaches a plateau, making it economically inefficient.), and PGE2 (50 ng/mL: approximately 30 ng/ml to 70 ng/ml is preferable. If the concentration is lower, the stimulation is too weak, resulting in slower differentiation. Conversely, if the concentration is higher, the differentiation rate reaches a plateau, making it economically inefficient.) are added to the liquid culture medium in the culture vessel. It is preferable to add these four factors on day 6 from the start of culture. In response to the stimulation from these added factors, immature dendritic cells undergo maturation and become mature dendritic cells. Notably, OK-432 is commercially available as Picibanil, manufactured by Chugai Pharmaceutical Co., Ltd., and PGE2 refers to Prostaglandin E2 (PGE2). The substances used to stimulate maturation are preferably GM-CSF, IL-4, OK-432, and PGE2, but are not limited thereto.

### Embodiment 1: (5) NKT-Stimulating Dendritic Cell Induction Step: Final Inspection

After obtaining mature dendritic cells, a sterility test, endotoxin test, and mycoplasma exclusion test will be conducted to confirm whether the obtained cells are safe for injection administration to humans. Additionally, surface marker measurement is performed to evaluate the quality of the dendritic cells. These quality confirmation tests may be supplemented with additional tests for other items as appropriate. If necessary, such as when the immediate administration to the beneficiary is not planned or is planned to be divided into multiple doses, the manufactured dendritic cells are collected and prepared to be 0.5×10⁷ cells/ml/vial (0.5×10⁶ cells/ml/vial when whole blood is used as raw material) using cryopreservation solution and cryopreserved in liquid nitrogen. A vial is one type of container used for injectable formulations and is classified as a sealed container under the Japanese Pharmacopoeia. To obtain the desired effect, it is preferable that the dendritic cell concentration meets or exceeds the aforementioned level. This is the method for producing the NKT cell-stimulating dendritic cell preparation of the present invention.

### Embodiment 1: Effect of the NKT Cell-Stimulating Dendritic Cell Preparation

When the NKT cell-stimulating dendritic cell preparation of the present invention is used on the beneficiary from whom the blood was collected, the preparation of the present invention stimulates and activates Vα24-NKT cells within the beneficiary's body to induce the production of IFN-γ, a type of cytokine, thereby exerting the adjuvant effect. NK cells are strongly activated by this adjuvant effect, leading to a cytotoxic response that attacks cancer cells or cells other than normal cells. Additionally, the activated NKT cells promote the maturation of immature dendritic cells present in the body, thereby enhancing the acquired immune system. By activating both the innate immune system and the acquired immune system, the preparation of the present invention is effective not only for cancer treatment but also for cancer prevention and for preventing disease by enhancing immunity in healthy individuals.

Next, the following is a description of the effects of reintroducing the NKT-cell stimulating dendritic cell preparation of the invention into the body of the beneficiary from whom the monocytes were collected for producing said preparation.

### Embodiment 1: Action of the Present Preparation: Reintroduction Into the Body, Intravenous Infusion

The present preparation, which comprises dendritic cells matured after pulsed with α-Galactosylceramide when they were immature dendritic cells, may be reintroduced into the beneficiary's body by intravenous infusion or other methods.

### Embodiment 1: Action of the Present Preparation: Activation of Vα24-NKT Cells

Passing through blood vessels in the body, the dendritic cells in the preparation of the present invention bind the incorporated glycolipid antigen α-Galactosylceramide to the ligand, CD1d, and present it to Vα24-NKT cells in the body. The Vα24-NKT cells in the body become activated when α-Galactosylceramide binds to their receptors (through antigen presentation).

Embodiment 1: Action of the Present Preparation: Activated NKT cells: TNF-α Production, Angiogenesis Inhibition

Activated Vα24-NKT cells produce TNF-α (tumor necrosis factor-α, a type of tumor necrosis factor), which stimulates immature dendritic cells (immature DCs). At the same time, the activated Vα24-NKT cells exerts an angiogenesis-inhibitory effect on cancer cells by TNF-α.

### Embodiment 1: Action of the Present Preparation: Maturation of Dendritic Cells and Activation of the Acquired Immune System

Immature dendritic cells phagocytose neoantigens, which are cancer antigens released by cancer cells or dead cancer cells. Alternatively, immature dendritic cells stimulated by TNF-α from activated Vα24-NKT cells phagocytose neoantigens. The dendritic cells that have phagocytosed neoantigens undergo maturation, further promoted by the stimulatory effect of TNF-α. Killer T cells and helper T cells receive antigen presentation from the matured dendritic cells as stimulation. The helper T cells that have received antigen presentation stimulate B cells via cytokines. The stimulated B cells produce and release cancer cell-specific antibodies.

### Embodiment 1: Action of the Present Preparation: Apoptosis, Cytotoxic Activity

NK cells stimulated by IFN-γ produced by activated Vα24-NKT cells become activated. These activated NK cells and activated Vα24-NKT cells attack cancer cells (or pathogens) that are not the beneficiary's own cells by killing (exerting cytotoxic activity) or promoting apoptosis (innate immune system). Meanwhile, the antibodies released by B cells bind to cancer cells. Killer T cells attack cancer cells (or pathogens) to which antibodies bind by puncturing the cell membrane to induce cancer cell necrosis (cytotoxic activity) or by producing substances such as TNF-β (tumor necrosis factor, a type of cytokine) to promote apoptosis (acquired immune system). The attack by activated NK cells and activated Vα24-NKT cells (innate immune response) occurs within 1 to 2 days after intravenous administration of the present preparation. In contrast, the attack by killer T cells (adaptive immune response) requires longer time and occurs 7 to 14 days later, as it occurs after receiving antigen presentation by dendritic cells.

Although killer T cells and helper T cells that have attacked cancer cells cannot survive for a long period of time, some of the aforementioned T cells exist as memory killer T cells and memory helper T cells in lymph nodes, etc. (It is believed that T cells with memory are replicated and exchanged to be maintained). Even after the cancer cells have been eliminated, if cancer cells with the same antigen reappear, the memory helper T cells, retaining their previous memory, quickly instruct immune cells to attack. This enables killer T cells to respond rapidly. In addition to T cells, B cells that have released antibodies also exist as memory B cells. And can promptly release antibodies in case of recurrence.

### Effects

By producing NKT cell-stimulating dendritic cells, when they are reintroduced to the beneficiary's body, these cells are expected to activate NK cells from the pathway of the innate immune system and indirectly activate helper T cells, killer T cells, and B cells by stimulating dendritic cells. Rather than solely targeting specific cancers, the present preparation strengthens overall immune function, it is expected that beneficiaries who have become weakened due to cancer onset will be able to prevent the onset of diseases other than cancer, and even if they do contract such diseases, their symptoms will be milder, and their recovery will be faster. The present preparation invention is effective not only for treating cancer but also for preventing cancer and for preventing other diseases by enhancing immunity. Furthermore, the present dendritic cell preparation is a preparation cultured from the beneficiary's autologous cells and can be used only for said beneficiary, making it free from rejection reactions and highly safe.

### <Embodiment 2: Overview> Primarily Corresponding to Claim 2

In Embodiment 2, the pulsing step (4) is carried out on day 3 or day 4, wherein day 0 is defined as the day on which the predetermined factors are added in the differentiation step (3), based on the method for producing the dendritic cell preparation of Embodiment 1.

### Embodiment 2: (4) Pulsing Step: Pulsing of α-Galactosylceramide

In Embodiment 2, the pulsing timing of α-Galactosylceramide is earlier than in Embodiment 1. When day 0 is defined as the day on which GM-CSF and IL-4 are added as predetermined factors in the differentiation step (3), α-Galactosylceramide is pulsed into the liquid culture medium in the culture vessel, where the immature dendritic cells are predominant, on either day 3 or day 4. As in Embodiment 1, α-Galactosylceramide used in this embodiment was KRN7000, a code name by Kyowa Kirin Co., Ltd.

### Embodiment 2: (4) Pulsing Step: Optimal Pulsing Timing

The dendritic cell preparation produced by the production method of the present invention is expected to stimulate and activate Vα24-NKT cells, thereby inducing the production of cytokines such as IFN-γ and activating various players of the immune system. To evaluate the efficacy of the preparation, non-adherent cells (including lymphocytes (T cells, B cells, NK cells, and NKT cells) or the like) removed in the non-adherent cell removal step (2) in the production method and separately cultured and preserved are added to the completed preparation and co-cultured to examine the ratio of IFN-γ-producing NKT cells to the total number of lymphocytes. The relationship between the pulsing timing and the NKT cell ratio after co-culture is analyzed to determine the optimal pulsing timing. The above-described pulsing timing was determined in this manner. The use of non-adherent cells is solely to avoid additional physical burden on the beneficiary, and no other intention is implied. Therefore, peripheral blood may be newly collected from the beneficiary, and the dendritic cell preparation produced by the production method of the present invention may be added to the collected peripheral blood to compare and confirm the effects in the same way. Additionally, co-culture was carried out to observe differences in the degree of increase in the NKT cell proportion relative to total lymphocytes, and these observations revealed that the proliferation rate of NKT cells (presumably through self-renewal and proliferation) is influenced by the dendritic cell preparation of the present invention. Therefore, although step (5) in claim 1 of the present application is simply described as “NKT-stimulating dendritic cell induction step”, it can also be referred to as “NKT proliferation-stimulating dendritic cell induction step”.

In this embodiment, all time references below are expressed in days when day 0 is defined as the day on which the predetermined factors (e.g., GM-CSF and IL-4) are added to induce the differentiation of monocytes into immature dendritic cells. After the completion of the preparation, co-culture was conducted by adding non-adherent cells to confirm the efficacy of the preparation. To verify the optimal pulsing timing, the collected monocytes were divided into three groups, and three samples of dendritic cell preparations with different pulsing timings of α-Galactosylceramide were prepared. One sample was pulsed with α-Galactosylceramide (concentration 100 ng/ml) on day 2 (48 hours) (condition 1), one sample was pulsed on day 4 (96 hours) (condition 2), and one sample was pulsed on the day 5 (120 hours) (condition 3) after the addition of the predetermined factors.

To confirm the efficacy of the present preparation, non-adherent cells are added on day 6 to start the co-culture, with day 0 defined as the day when the predetermined factors for inducing the differentiation of monocytes into immature dendritic cells are added. The ratio of NKT cell count to total lymphocyte count was analyzed at three time points: on the day of the start of co-culture (day 0 from the start of co-culture), on day 7 from the day of the start of co-culture, and on day 14 from the day of the start of co-culture.

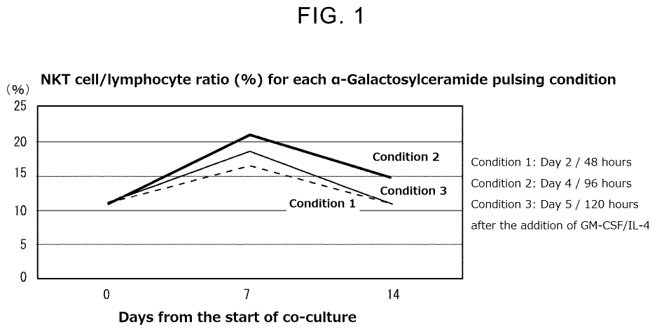

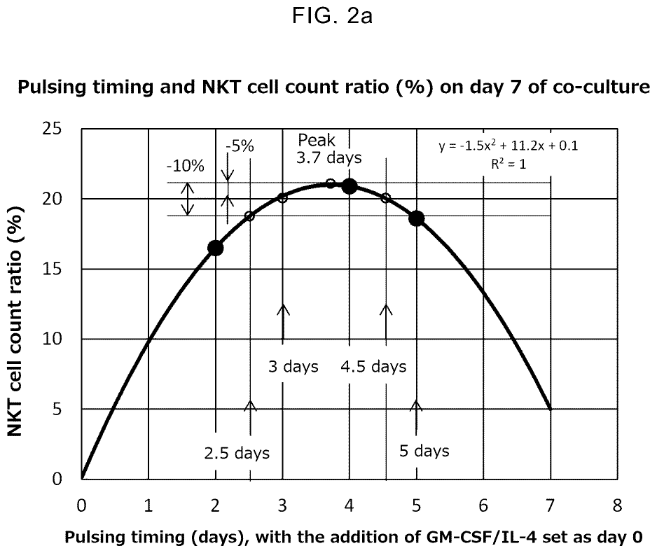

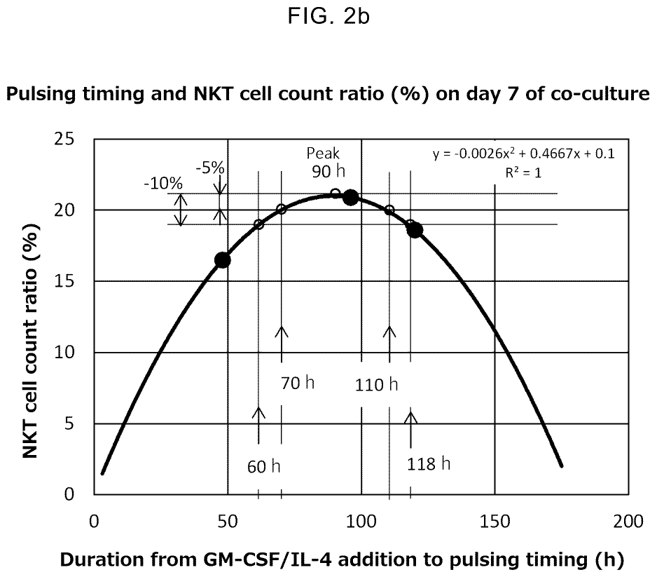

FIG. 1 shows the conditions of pulsing timing of α-Galactosylceramide (Conditions 1 to 3) and the investigation results on the ratio of IFN-γ-producing NKT cells (those originally contained in the non-adherent cells) to the total lymphocyte count at each time point after co-culture. These investigation results were obtained using a flow cytometer. In all three conditions, the ratio of NKT cell count to total lymphocyte count reached its maximum on day 7 after the start of the co-culture. Among the three conditions, the highest NKT cell count ratio (%) was observed when pulsing was conducted on day 4, with day 0 defined as the day when the predetermined factors were added to induce the differentiation of monocytes into immature dendritic cells. The relationship between said highest NKT cell count ratio (%) on day 7 after co-culture and the pulsing timing (days) with day 0 defined as the day of addition of the predetermined factors, is shown in the scatter plot in FIG. 2a. FIG. 2a shows an approximate curve fitted using a quadratic equation which is also extrapolated before and after. The coefficient of determination of this approximate curve is 1, which is considered to be sufficiently reliable. The peak of the curve is located between day 3 and day 4 of pulsing timing (calculated as day 3.7 based on the approximate equation). The preferred range is within −10% of the peak value, which corresponds to approximately day 2.5 to day 5. Since the lifespan of dendritic cells is approximately a few days to one week, if the manufacturing process (the manufacturing process refers to the period of steps from monocyte collection or from the addition of the predetermined factors to induce differentiation of monocytes into immature dendritic cells, until their maturation into mature dendritic cells, specifically the period up to the addition of GM-CSF, IL-4, OK-432, and PGE2 for inducing mature dendritic cells (the time required for complete maturation after this step is approximately 10 to 20 hours, and this duration may also be included in the manufacturing process.). In this experiment, while the pulsing timing of α-Galactosylceramide varied among Conditions 1 to 3, the addition of GM-CSF, IL-4, OK-432, and PGE2 was performed simultaneously for all conditions.) takes too long in the present production method, the usable period of the preparation will be shortened. Therefore, considering that the formulation can be completed within one week from the start of culture, the preferable pulsing timing is between day 3 to day 4.5 corresponding to the range within −5% of the peak value, and it is preferable to pulse on day 3 or day 4. The most preferred pulsing timing is on day 4.

When dendritic cells are pulsed with α-Galactosylceramide before maturation, the produced preparation can achieve maximum efficacy in the beneficiary's body when pulsing is performed on either day 4 or day 3 after the addition of the predetermined factors (e.g., GM-CSF and IL-4) for the differentiation of monocytes into immature dendritic cells.

### <Embodiment 3: Overview> Primarily Corresponding to Claim 3

In Embodiment 3, the pulsing step (4) is carried out within a period of 60 hours to 108 hours after addition of the predetermined factors in the differentiation step (3), based on the method for producing the dendritic cell preparation of Embodiment 1.

### Embodiment 3: (4) Pulsing Step: Pulsing of α-Galactosylceramide

In Embodiment 3, the pulsing timing of α-Galactosylceramide is earlier than in Embodiment 1. α-Galactosylceramide is pulsed within a period of 60 hours to 108 hours after addition of GM-CSF and IL-4 as the predetermined factors in the differentiation step. As in Embodiment 1, α-Galactosylceramide used in this embodiment was KRN7000, a code name by Kyowa Kirin Co., Ltd.

### Form 3: (4) Pulsing Step: Optimal Pulsing Timing

The dendritic cell preparation produced by the production method of the present invention is expected to stimulate and activate Vα24-NKT cells, thereby inducing the production of cytokines such as IFN-γ and activating various players of the immune system. To evaluate the efficacy of the preparation, non-adherent cells (including lymphocytes (T cells, B cells, and NK cells) or the like) removed in the non-adherent cell removal step (2) in the production method are added to the completed preparation and co-cultured to examine the ratio of IFN-γ-producing NKT cell count. The relationship between the pulsing timing and the NKT cell ratio after co-culture is analyzed to determine the optimal pulsing timing in the same manner as in Embodiment 2 above. The above-described pulsing timing was determined in this manner. The use of non-adherent cells is solely to avoid additional physical burden on the beneficiary, and no other intention is implied. Therefore, peripheral blood may be newly collected from the beneficiary, and the dendritic cell preparation produced by the production method of the present invention may be added to the collected peripheral blood to compare and confirm the effects in the same way. Additionally, co-culture was carried out to observe differences in the degree of increase in the NKT cell proportion relative to total lymphocytes, and these observations revealed that the proliferation rate of NKT cells (presumably through self-renewal and proliferation) is influenced by the dendritic cell preparation of the present invention. Therefore, although step (5) in claim 1 of the present application is simply described as “NKT-stimulating dendritic cell induction step”, it can also be referred to as “NKT proliferation-stimulating dendritic cell induction step”.

In this embodiment, all time references below are expressed in hours, with 0 hours defined as the time when the predetermined factors (e.g., GM-CSF and IL-4) are added to induce the differentiation of monocytes into immature dendritic cells. After the completion of the preparation, co-culture was conducted by adding non-adherent cells to confirm the efficacy of the preparation. To verify the optimal pulsing timing, the production method based on Embodiment 1 was used, and the collected monocytes were divided into three groups, preparing three types of dendritic cell preparation samples that differed only in the pulsing timing of α-Galactosylceramide. One sample was pulsed with α-Galactosylceramide (concentration 100 ng/ml) at 48 hours (day 2) (Condition 1), one sample was pulsed at. 96 hours (day 4) (Condition 2), and one sample was pulsed at 120 hours (day 5) (Condition 3) after the addition of the predetermined factors.

To confirm the efficacy of the present preparation, non-adherent cells were added at 144 hours (day 6) after the addition of predetermined factors for inducing the differentiation of monocytes into immature dendritic cells, to start the co-culture. The ratio of NKT cell count to total lymphocyte count was analyzed at three time points: on the day of the start of co-culture (day 0 from the start of co-culture), on day 7 from the start of co-culture, and on day 14 from the start of co-culture.

FIG. 1 shows the conditions of pulsing timing of α-Galactosylceramide (Conditions 1 to 3) and the investigation results on the ratio of IFN-γ-producing NKT cells (those originally contained in the non-adherent cells) to the total lymphocyte count at each time point after co-culture. These investigation results were obtained using a flow cytometer. In all three conditions, the ratio of NKT cell count to total lymphocyte count reached its maximum on day 7 after the start of co-culture. Among the three conditions, the highest NKT cell count ratio (%) was observed when pulsing was conducted at 96 hours (day 4) after the addition of the predetermined factors for inducing the differentiation of monocytes into immature dendritic cells. The relationship between said highest NKT cell count ratio (%) on day 7 after co-culture and the pulsing timing is shown in the scatter plot in FIG. 2b. FIG. 2b shows an approximate curve fitted using a quadratic equation, which is also extrapolated before and after the observed data points. The coefficient of determination of this approximate curve is 1, which is considered to be sufficiently reliable. The peak of the curve is located between pulsing timings of 70 hours and 96 hours after the addition (calculated as 90 hours based on the approximate equation).

The preferred range is within −10% of the peak value, which corresponds to approximately 60 hours to 118 hours after the addition. Since the lifespan of dendritic cells is approximately a few days to one week (up to 168 hours), if the manufacturing process (the manufacturing process refers to the period of steps from monocyte collection or from the addition of the predetermined factors for inducing differentiation of monocytes into immature dendritic cells, until their maturation into mature dendritic cells, specifically the period up to the addition of GM-CSF, IL-4, OK-432, and PGE2 for inducing mature dendritic cells (the time required for complete maturation after this step is approximately 10 to 20 hours, and this duration may also be included in the manufacturing process). In this experiment, while the pulsing timing of α-Galactosylceramide varied among Conditions 1 to 3, the addition of GM-CSF, IL-4, OK-432, and PGE2 was performed simultaneously for all conditions.) takes too long in the present production method, the usable period of the preparation will be shortened. Therefore, considering that the preparation can be completed within one week from the start of culture, it is preferable to pulse within the period of 60 hours to 108 hours after the addition. More preferably, pulsing is performed within the period of 70 hours to 108 hours after the addition, which corresponds to the range within −5% of the peak value.

When dendritic cells are pulsed with α-Galactosylceramide before maturation, the produced preparation can achieve maximum efficacy in the beneficiary's body when pulsing is performed within the period of 60 hours to 108 hours after the addition of the predetermined factors (e.g., GM-CSF and IL-4) for the differentiation of monocytes into immature dendritic cells.

### <Embodiment 4: Overview> Primarily Corresponding to Claim 4

In Embodiment 4, the pulsing step (4) is carried out by day 4 or day 5, wherein day 0 is defined as the day on which the monocytes collected from a beneficiary are placed in the culture vessel in the adhesion step (1), based on the method for producing the dendritic cell preparation of Embodiment 1.

### Embodiment 4: (4) Pulsing Step: Pulsing of α-Galactosylceramide

In Embodiment 4, the pulsing timing of α-Galactosylceramide is earlier than in Embodiment 1. α-Galactosylceramide is pulsed by day 4 or day 5, wherein day 0 is defined as the day on which the monocytes collected from a beneficiary are placed in the culture vessel. As in Embodiment 1, α-Galactosylceramide used in this embodiment was KRN7000, a code name by Kyowa Kirin Co., Ltd.

### Form 4: (4) Pulsing Step: Optimal Pulsing Timing

The dendritic cell preparation produced by the production method of the present invention is expected to stimulate and activate Vu24-NKT cells, thereby inducing the production of IFN-γ and activating various players of the immune system. To evaluate the efficacy of the preparation, non-adherent cells (including lymphocytes (T cells, B cells, and NK cells) or the like) removed in the non-adherent cell removal step (2) in the production method are added to the completed preparation and co-cultured to examine the ratio of IFN-γ-producing NKT cell count. The relationship between the pulsing timing and the NKT cell count ratio after co-culture is analyzed to determine the optimal pulsing timing. The above-described pulsing timing was determined in this manner. The use of non-adherent cells is solely to avoid additional physical burden on the beneficiary, and no other intention is implied. Therefore, peripheral blood may be newly collected from the beneficiary, and the dendritic cell preparation produced by the production method of the present invention may be added to the collected peripheral blood to compare and confirm the effects in the same way. Additionally, co-culture was carried out to observe differences in the degree of increase in the NKT cell proportion relative to total lymphocytes, and these observations revealed that the proliferation rate of NKT cells (presumably through self-renewal and proliferation) is influenced by the dendritic cell preparation of the present invention. Therefore, although step (5) in claim 1 of the present application is simply described as “NKT-stimulating dendritic cell induction step”, it can also be referred to as “NKT proliferation-stimulating dendritic cell induction step”.

In this embodiment, all time references below are expressed in days, with starting point set as the start of culture, defined as the time when monocytes collected from the beneficiary are placed into the culture vessel. After the completion of the preparation, co-culture was conducted by adding non-adherent cells to confirm the efficacy of the preparation. To verify the optimal pulsing timing, the production method based on the production method of Embodiment 1 was used, and the collected monocytes were divided into three groups, and three types of dendritic cell preparation samples that differed only in the pulsing timing of α-Galactosylceramide were prepared. One sample was pulsed with α-Galactosylceramide (concentration 100 ng/ml) at 3 days (72 hours) (Condition 1), one sample was pulsed at 5 days (120 hours) (Condition 2), and one sample was pulsed at 6 days (144 hours) (Condition 3) after the monocytes were placed into the culture vessel. In this experiment, GM-CSF and IL-4 were added 24 hours after the monocytes were placed into the culture vessel, as the predetermined factors to induce the differentiation of monocytes into immature dendritic cells.

To confirm the efficacy of the present preparation, non-adherent cells are added on day 7, wherein day 0 is defined as the day on which the monocytes collected from the beneficiary are placed into the culture vessel. The ratio of NKT cell count to total lymphocyte count was analyzed at three time points: on the day of the start of co-culture (day 0 from the start of co-culture), on day 7 from the start of co-culture, and on day 14 from the start of co-culture.

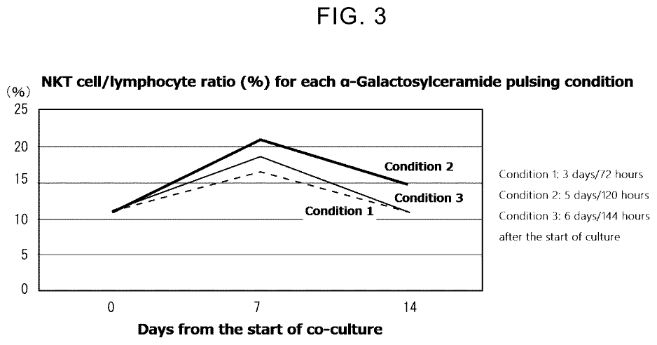

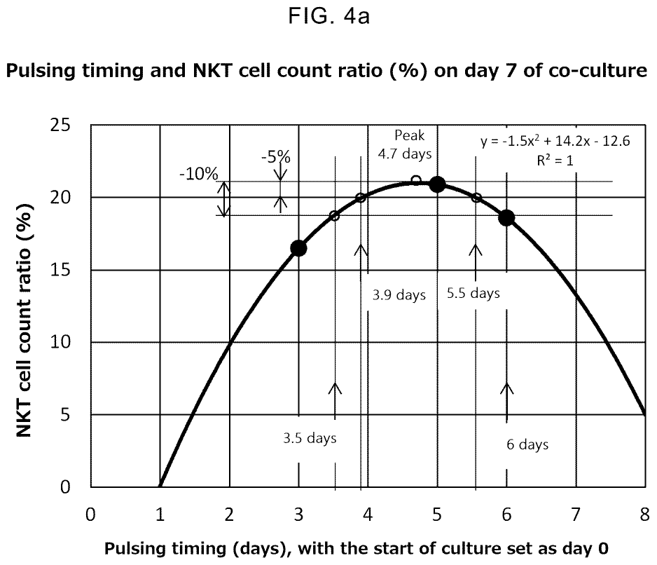

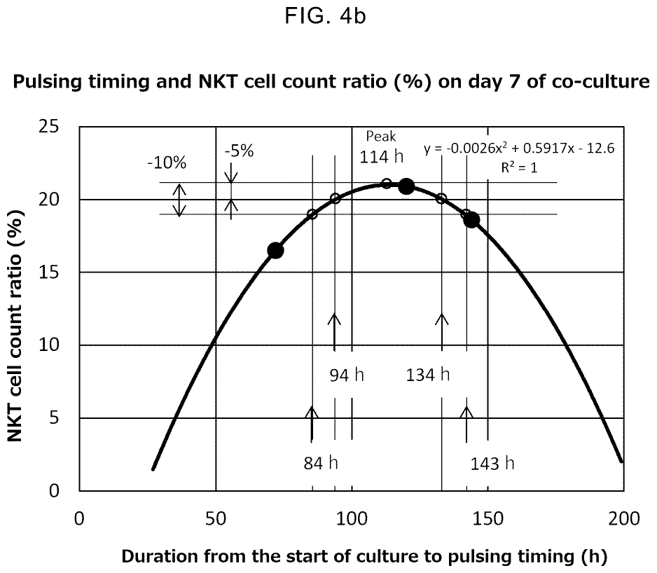

FIG. 3 shows the conditions of pulsing timing of α-Galactosylceramide (Conditions 1 to 3) and the investigation results on the ratio of IFN-γ-producing NKT cells (those originally contained in the non-adherent cells) to the total lymphocyte count at each time point after co-culture. These investigation results were obtained using a flow cytometer. In all three conditions, the ratio of NKT cell count to total lymphocyte count reached its maximum on day 7 after the start of co-culture. Among the three conditions, the highest NKT cell count ratio (%) was observed when pulsing was conducted on day 5 after the start of culture. The relationship between said highest NKT cell count ratio (%) on day 7 after co-culture and the pulsing timing is shown in the scatter plot in FIG. 4a. FIG. 4a shows an approximate curve fitted using a quadratic equation, which is also extrapolated before and after the observed data points. The coefficient of determination of this approximate curve is 1, which is considered to be sufficiently reliable. The peak of the curve is located between day 4 and day 5, with day 0 defined as the start of culture (calculated as day 4.7 based on the approximate equation). The preferred range is within −10% of the peak value, which corresponds to approximately day 3.5 to day 6. Since the lifespan of dendritic cells is approximately a few days to one week, if the manufacturing process (the manufacturing process refers to the period of steps from monocyte collection or from the addition of predetermined factors to induce differentiation of monocytes into immature dendritic cells, until their maturation into mature dendritic cells, specifically the period up to the addition of GM-CSF, IL-4, OK-432, and PGE2 for inducing mature dendritic cells (the time required for complete maturation after this step is approximately 10 to 20 hours, and this duration may also be included in the manufacturing process). In this experiment, while the pulsing timing of α-Galactosylceramide varied among Conditions 1 to 3, the addition of GM-CSF, IL-4, OK-432, and PGE2 was performed simultaneously for all conditions.) takes too long in the present production method, the usable period of the preparation will be shortened. Therefore, considering that the preparation can be completed within one week from the start of culture, the more preferable pulsing range is between day 3.9 and day 5.5, corresponding to the range within −5% of the peak. The most preferred pulsing timing is on day 5.

When dendritic cells are pulsed with α-Galactosylceramide before maturation, the produced preparation can achieve maximum efficacy in the beneficiary's body when pulsing is performed on either day 4 or day 5, with day 0 defined as the day on which monocytes collected from the beneficiary are placed into the culture vessel.

### <Embodiment 5: Overview> Primarily Corresponding to Claim 5

In Embodiment 5, the pulsing step (4) is carried out within the period of 84 hours to 132 hours after the mononuclear cells collected from the beneficiary are placed into the culture vessel in the adhesion step (1), based on the method for producing the dendritic cell preparation of Embodiment 1.

### Embodiment 5: (4) Pulsing Step: Pulsing of α-Galactosylceramide

The pulsing timing of α-Galactosylceramide in Embodiment 5 is earlier than in Embodiment 1. α-Galactosylceramide is pulsed between 84 hours and 132 hours after the monocytes collected from the beneficiary are placed into the culture vessel. As in Embodiment 1, the α-Galactosylceramide used was KRN7000, a code name by Kyowa Kirin Co., Ltd.

### Embodiment 5: (4) Pulsing Step: Optimal Pulsing Timing

The dendritic cell preparation produced by the production method of the present invention is expected to stimulate and activate Vα24-NKT cells, thereby inducing the production of IFN-γ and activating various players of the immune system. To evaluate the efficacy of the preparation, non-adherent cells (including lymphocytes (T cells, B cells, and NK cells) or the like) removed in the non-adherent cell removal step (2) in the production method are added to the completed preparation and co-cultured to examine the ratio of IFN-γ-producing NKT cell count. As in Embodiment 4, the relationship between the pulsing timing and the NKT cell count ratio after co-culture is analyzed to determine the optimal pulsing timing. The above-described pulsing timing was determined in this manner. The use of non-adherent cells is solely to avoid additional physical burden on the beneficiary, and no other intention is implied. Therefore, peripheral blood may be newly collected from the beneficiary. and the dendritic cell preparation produced by the production method of the present invention may be added to the collected peripheral blood to compare and confirm the effects in the same way. Additionally, co-culture was carried out to observe differences in the degree of increase in the NKT cell proportion relative to total lymphocytes, and these observations revealed that the proliferation rate of NKT cells (presumably through self-renewal and proliferation) is influenced by the dendritic cell preparation of the present invention. Therefore, although step (5) in claim 1 of the present application is simply described as “NKT-stimulating dendritic cell induction step”, it can also be referred to as “NKT proliferation-stimulating dendritic cell induction step”.

In this embodiment, all time references below are expressed in hours, with the starting point set as the start of culture, defined as the time when monocytes collected from the beneficiary are placed into the culture vessel. After the completion of the preparation, co-culture was conducted by adding non-adherent cells to confirm the efficacy of the preparation. To verify the optimal pulsing timing, the production method based on the production method of Embodiment 1 was used, and the collected monocytes were divided into three groups, and three types of dendritic cell preparation samples that differed only in the pulsing timing of α-Galactosylceramide were prepared. One sample is pulsed with o-Galactosylceramide (concentration 100 ng/ml) at 72 hours (3 days) (Condition 1), one sample is pulsed at 120 hours (5 days) (Condition 2), and one sample is pulsed at 144 hours (6 days) (Condition 3), after the monocytes are placed into the culture vessel. In this experiment, GM-CSF and IL-4 were added 24 hours after the monocytes were placed into the culture vessel, as the predetermined factors to induce the differentiation of monocytes into immature dendritic cells.

To confirm the efficacy of the present preparation, non-adherent cells are added at 168 hours after the mononuclear cells collected from the beneficiary are placed into the culture vessel to start the co-culture. The ratio of NKT cell count to total lymphocyte count was analyzed at three time points: on the day of the start of co-culture (day 0 from the start of co-culture), on day 7 from the start of co-culture, and on day 14 from the start of co-culture.

FIG. 3 shows the conditions of pulsing timing of α-Galactosylceramide (Conditions 1 to 3) and the investigation results on the ratio of IFN-γ-producing NKT cells (those originally contained in the non-adherent cells) to the total lymphocyte count at each time point after co-culture. These investigation results were obtained using a flow cytometer. In all three conditions, the ratio of NKT cell count to total lymphocyte count reached its maximum on day 7 after the start of co-culture. Among the three conditions, these investigation results were obtained using a flow cytometer. The highest NKT cell count ratio (%) on day 7 after co-culture was observed when pulsing was conducted on day 5 after the start of culture. The relationship between the maximum NKT cell count ratio (%) on day 7 after co-culture and the pulsing timing is shown in the scatter plot in FIG. 4b. FIG. 4b shows an approximate curve fitted using a quadratic equation, which is also extrapolated before and after the observed data points. The coefficient of determination of this approximate curve is 1, which is considered to be sufficiently reliable. The peak of the curve is located between 96 hours (day 4, with day 0 defined as the start of culture) and 120 hours (day 5, with day 0 defined as the start of culture), approximately at 114 hours after the start of culture. The preferred range is within −10% of the peak value, which corresponds to approximately 84 hours to 143 hours after the start of culture. Since the lifespan of dendritic cells is approximately a few days to one week (168 hours), if the manufacturing process (the manufacturing process refers to the period of steps from monocyte collection or from the addition of predetermined factors to induce differentiation of monocytes into immature dendritic cells until their maturation into mature dendritic cells, specifically the period up to the addition of GM-CSF, IL-4, OK-432, and PGE2 for inducing mature dendritic cells (the time required for complete maturation after this step is approximately 10 to 20 hours, and this duration may also be included in the manufacturing process). In this experiment, while the pulsing timing of α-Galactosylceramide varied among Conditions 1 to 3, the addition of GM-CSF, IL-4, OK-432, and PGE2 was performed simultaneously for all conditions.) takes too long in the present production method, the usable period of the preparation will be shortened. Therefore, to complete the preparation within one week from the start of culture, it is preferable to pulse within the period of 84 hours to 132 hours after the start of culture. More preferably, pulsing should be conducted within the period of 94 hours to 132 hours after the start of culture, which corresponds to the range within −5% of the peak.

When dendritic cells are pulsed with α-Galactosylceramide before maturation, the produced preparation can achieve maximum efficacy in the beneficiary's body when pulsing is performed within the period of 84 hours to 132 hours after the monocytes collected from the beneficiary are placed into the culture vessel.

### <Embodiment 6: Overview> Primarily Corresponding to Claim 6

In Embodiment 6, the pulsing step (4) is carried out on day 4 or day 5, with day 0 defined as the day on which non-adherent cells, including monocytes, which are cells other than those adhered to the inner surface of the culture vessel, are removed in the non-adherent cell removal step (2), based on the method for producing the dendritic cell preparation of Embodiment 1.

### Embodiment 6: (4) Pulsing Step: Pulsing of α-Galactosylceramide

The pulsing timing of α-Galactosylceramide in Embodiment 6 is earlier than in Embodiment 1. α-Galactosylceramide is pulsed by day 4 or day 5, with day 0 defined as the day on which non-adherent cells, including monocytes, which are cells other than those adhered to the inner surface of the culture vessel, are removed in the non-adherent cell removal step (2). As in Embodiment 1, α-Galactosylceramide used in this embodiment was KRN7000, a code name by Kyowa Kirin Co., Ltd.

### Embodiment 6: (4) Pulsing Step: Optimal Pulsing Timing

The dendritic cell preparation produced by the production method of the present invention is expected to stimulate and activate Vα24-NKT cells, thereby inducing the production of IFN-γ and activating various players of the immune system. To evaluate the efficacy of the preparation, non-adherent cells (including lymphocytes (T cells, B cells, and NK cells) or the like) removed in the non-adherent cell removal step (2) in the production method are added to the completed preparation and co-cultured to examine the ratio of IFN-γ-producing NKT cell count. The relationship between the pulsing timing and the NKT cell count ratio after co-culture is analyzed to determine the optimal pulsing timing. The above-described pulsing timing was determined in this manner. The use of non-adherent cells is solely to avoid additional physical burden on the beneficiary, and no other intention is implied. Therefore, peripheral blood may be newly collected from the beneficiary, and the dendritic cell preparation produced by the production method of the present invention may be added to the collected peripheral blood to compare and confirm the effects in the same way. Additionally, co-culture was carried out to observe differences in the degree of increase in the NKT cell proportion relative to total lymphocytes, and these observations revealed that the proliferation rate of NKT cells (presumably through self-renewal and proliferation) is influenced by the dendritic cell preparation of the present invention. Therefore, although step (5) in claim 1 of the present application is simply described as “NKT-stimulating dendritic cell induction step”, it can also be referred to as “NKT proliferation-stimulating dendritic cell induction step”.

In this embodiment, all time references below are expressed in days, with the starting point set as the time of removal of non-adherent cells, including monocytes, which are cells other than those adhered to the inner surface of the culture vessel, in the non-adherent cell removal step (2). After the completion of the preparation, co-culture was conducted by adding the non-adherent cells to confirm the efficacy of the preparation. To verify the optimal pulsing timing, the production method based on the production method of Embodiment 1 was used, and the collected monocytes were divided into three groups, and three types of dendritic cell preparation samples that differed only in the pulsing timing of α-Galactosylceramide were prepared. One sample was pulsed with α-Galactosylceramide (concentration 100 ng/ml) at 3 days (72 hours) (Condition 1), one sample was pulsed at 5 days (120 hours) (Condition 2), and one sample was pulsed at 6 days (144 hours) (Condition 3), after the removal of non-adherent cells including monocytes, which are cells other than those adhered to the inner surface of the culture vessel, in the non-adherent cell removal step (2). In this experiment, GM-CSF and IL-4 were added 24 hours after the removal of non-adherent cells including monocytes, which are cells other than those adhered to the inner surface of the culture vessel, in the non-adherent cell removal step (2), as the predetermined factors for inducing the differentiation of monocytes into immature dendritic cells.

To confirm the efficacy of the present preparation, non-adherent cells are added on day 7 to start the co-culture, with day 0 defined as the day on which non-adherent cells, including monocytes, which are cells other than those adhered to the inner surface of the culture vessel, were removed in the non-adherent cell removal step (2). The ratio of NKT cell count to lymphocyte count was analyzed at three time points: on the day of the start of co-culture (day 0 from the start of co-culture), on day 7 from the start of co-culture, and on day 14 from the start of co-culture.

FIG. 3 shows the conditions of pulsing timing of α-Galactosylceramide (Conditions 1 to 3) and the investigation results on the ratio of IFN-γ-producing NKT cells (those originally contained in the non-adherent cells) to the total lymphocyte count at each time point after co-culture. The phrase “after the start of culture” in the conditions on the right side of FIG. 3 should be replaced with “after the removal of non-adherent cells, including monocytes, which are cells other than those adhered to the inner surface of the culture vessel, in the non-adherent cell removal step (2).” FIG. 3 shows the results of the investigation obtained using a flow cytometer. In all three conditions, the ratio of NKT cell count to total lymphocyte count reached its maximum on day 7 after the start of co-culture. Among the three conditions, the highest NKT cell count ratio (%) was observed when pulsing was conducted on day 5 after the removal of the non-adherent cells. The relationship between said highest NKT cell count ratio (%) on day 7 after co-culture and the pulsing timing is shown in the scatter plot in FIG. 4a. FIG. 4a shows an approximate curve fitted using a quadratic equation, which is also extrapolated before and after the observed data points. The coefficient of determination of this approximate curve is 1, which is considered to be sufficiently reliable. The peak of the curve is located between day 4 and day 5 (calculated as day 4.7 based on the approximate equation), with day 0 defined as the day on which the non-adherent cells were removed. The preferred range is within −10% of the peak value, which corresponds to approximately day 3.5 to day 6. Since the lifespan of dendritic cells is approximately a few days to one week, if the manufacturing process (the manufacturing process refers to the period of steps from monocyte collection or from the addition of predetermined factors to induce differentiation of monocytes into immature dendritic cells, until their maturation into mature dendritic cells, specifically the period up to the addition of GM-CSF, IL-4, OK-432, and PGE2 for inducing mature dendritic cells (the time required for complete maturation after this step is approximately 10 to 20 hours, and this duration may also be included in the manufacturing process). In this experiment, while the pulsing timing of α-Galactosylceramide varied among Conditions 1 to 3, the addition of GM-CSF, IL-4, OK-432, and PGE2 was performed simultaneously for all conditions.) takes too long in the present production method, the usable period of the preparation will be shortened. Therefore, considering that the preparation can be completed within one week from the start of culture, the more preferable is between day 3.9 and day 5.5, corresponding to the range within −5% of the peak. The most preferred pulsing timing is on day 5.

When dendritic cells are pulsed with α-Galactosylceramide before maturation, the produced preparation can achieve maximum efficacy in the beneficiary's body when pulsing is performed on either day 4 or day 5, with day 0 defined as the day on which non-adherent cells, including monocytes, which are cells other than those adhered to the inner surface of the culture vessel, were removed in the non-adherent cell removal step (2).

### <Embodiment 7: Overview> Primarily Corresponding to Claim 7

In Embodiment 7, the pulsing step (4) is carried out within a period of 84 hours to 132 hours after the removal of non-adherent cells, including monocytes, which are cells other than those adhered to the inner surface of the culture vessel, in the non-adherent cell removal step (2), based on the method for producing the dendritic cell preparation of Embodiment 1.

### Embodiment 7: (4) Pulsing Step: Pulsing of α-Galactosylceramide

In Embodiment 7, the pulsing timing of α-Galactosylceramide is earlier than in Embodiment 1. α-Galactosylceramide is pulsed between 84 hours and 132 hours after the removal of non-adherent cells, including monocytes, which are cells other than those adhered to the inner surface of the culture vessel, in the non-adherent cell removal step (2). As in Embodiment 1, α-Galactosylceramide used in this embodiment was KRN7000, a code name by Kyowa Kirin Co., Ltd.

### Embodiment 7: (4) Pulsing Step: Optimal Pulsing Timing

The dendritic cell preparation produced by the production method of the present invention is expected to stimulate and activate Vα24-NKT cells, thereby inducing the production of IFN-γ and activating various players of the immune system. To evaluate the efficacy of the preparation, non-adherent cells (including lymphocytes (T cells, B cells, and NK cells) or the like) removed in the non-adherent cell removal step (2) in the production method are added to the completed preparation and co-cultured to examine the ratio of IFN-γ-producing NKT cell count. The relationship between the pulsing timing and the NKT cell count ratio after co-culture is analyzed to determine the optimal pulsing timing, in the same manner as Embodiment 6. The above-described pulsing timing was determined in this manner. The use of non-adherent cells is solely to avoid additional physical burden on the beneficiary, and no other intention is implied. Therefore, peripheral blood may be newly collected from the beneficiary, and the dendritic cell preparation produced by the production method of the present invention may be added to the collected peripheral blood to compare and confirm the effects in the same way. Additionally, co-culture was carried out to observe differences in the degree of increase in the NKT cell proportion relative to total lymphocytes, and these observations revealed that the proliferation rate of NKT cells (presumably through self-renewal and proliferation) is influenced by the dendritic cell preparation of the present invention. Therefore, although step (5) in claim 1 of the present application is simply described as “NKT-stimulating dendritic cell induction step”, it can also be referred to as “NKT proliferation-stimulating dendritic cell induction step”.

In this embodiment, all time references below are expressed in hours, with the starting point set as the time as the removal of non-adherent cells including monocytes, which are cells other than those adhered to the inner surface of the culture vessel, in the non-adherent cell removal step (2). After the completion of the preparation, co-culture was conducted by adding the non-adherent cells to confirm the efficacy of the preparation. To verify the optimal pulsing timing, the production method based on the production method of Embodiment 1 was used, and the collected monocytes were divided into three groups, and three types of dendritic cell preparation samples that differed only in the pulsing timing of α-Galactosylceramide were prepared. One sample was pulsed with α-Galactosylceramide (concentration 100 ng/ml) at 72 hours (3 days) (Condition 1), one sample was pulsed at 120 hours (5 days) (Condition 2), and one sample was pulsed at 144 hours (6 days) (Condition 3), after the removal of non-adherent cells including monocytes other than cells adhered to the inner surface of the culture vessel. In this experiment, GM-CSF and IL-4 were added 24 hours after the removal of non-adherent cells, including monocytes other than cells adhered to the inner surface of the culture vessel, as the predetermined factors to induce the differentiation of monocytes into immature dendritic cells.

To confirm the efficacy of the present preparation, non-adherent cells are added at 168 hours after the removal of non-adherent cells to start the co-culture. The ratio of NKT cell count to lymphocyte count was analyzed at three time points: on the day of the start of co-culture (day 0 from the start of co-culture), on day 7 from the start of co-culture, and on day 14 from the start of co-culture.

FIG. 3 shows the conditions of pulsing timing o-Galactosylceramide (Conditions 1 to 3) and the investigation results on the ratio of IFN-γ-producing NKT cells (those originally contained in the non-adherent cells) to the total lymphocyte count at each time point after co-culture. The phrase “after the start of culture” in the conditions on the right side of FIG. 3 should be replaced with “after the removal of non-adherent cells, including monocytes, which are cells other than those adhered to the inner surface of the culture vessel, in the non-adherent cell removal step (2).” FIG. 3 shows the results of the investigation obtained using a flow cytometer. In all three conditions, the ratio of NKT cell count to total lymphocyte count reached its maximum on day 7 after the start of co-culture. Among the 3 conditions, it is the results of an investigation obtained using a flow cytometer. The highest NKT cell count ratio (%) was observed on day 7 after the start of co-culture, when pulsing was conducted on day 5 after the removal of the non-adherent cells. The relationship between said highest NKT cell count ratio (%) on day 7 after co-culture and the pulsing timing is shown in the scatter plot in FIG. 4b. FIG. 4b shows an approximate curve fitted using a quadratic equation, which is also extrapolated before and after the observed data points. The coefficient of determination of this approximate curve is 1, which is considered to be sufficiently reliable. The peak of the curve is located between 96 hours (day 4, with day 0 defined as the start of culture) and 120 hours (day 5, with day 0 defined as the start of culture), approximately at 114 hours after the removal of non-adherent cells. The preferred range is within −10% of the peak value, which corresponds to approximately 84 hours to 143 hours after the removal of non-adherent cells. Since the lifespan of dendritic cells is approximately a few days to one week, if the manufacturing process (the manufacturing process refers to the period of steps from monocyte collection or from the addition of predetermined factors to induce differentiation of monocytes into immature dendritic cells, until their maturation into mature dendritic cells, specifically the period up to the addition of GM-CSF, IL-4, OK-432, and PGE2 for inducing mature dendritic cells (the time required for complete maturation after this step is approximately 10 to 20 hours, and this duration may also be included in the manufacturing process). In this experiment, while the pulsing timing of α-Galactosylceramide varied among Conditions 1 to 3, the addition of GM-CSF, IL-4, OK-432, and PGE2 was performed simultaneously for all conditions.) takes too long in the present production method, the usable period of the preparation will be shortened. Therefore, in order to complete the preparation within one week from the start of culture, it is preferable to pulse within the period of 84 to 132 hours after the removal of non-adherent cells. More preferably, pulsing is performed within the period of 94 to 132 hours after the removal of non-adherent cells, which corresponds to the range within −5% of the peak.

When dendritic cells are pulsed with α-Galactosylceramide before maturation, the produced preparation can achieve maximum efficacy in the beneficiary's body when pulsing is performed within the period of 84 hours to 132 hours after the removal of non-adherent cells.

### <Embodiment 8: Overview> Mainly Corresponding to Claim 8

The concentration of α-Galactosylceramide used for pulsing immature dendritic cells to induce the NKT-stimulating dendritic cells, which are dendritic cells that stimulate Vα24-NKT cell-stimulated, is 300 ng/ml to 1000 ng/ml. In the present embodiment, the pulsing timing of α-Galactosylceramide is described based on the day when day 0 is defined as the day on which the monocytes collected from a beneficiary are placed in the culture vessel. In the present embodiment, the pulsing timing of α-Galactosylceramide is set to 120 hours after the start of culture, based on Embodiment 5. However, the pulsing timing may also be between 84 hours and 132 hours after the start of culture. Alternatively, as explained in Embodiment 4, pulsing may be performed 4 or 5 days after the start of culture, with day 0 defined as the day on which monocytes are placed into the culture vessel and culture is started. Alternatively, as explained in Embodiments 2 and 3, pulsing may be performed on day 3 or day 4, with day 0 defined as the day on which the predetermined factors are added in the differentiation step, or within the period of 60 to 108 hours after the addition of the predetermined factors. Alternatively, as described in Embodiments 6 and 7, pulsing may be performed on day 4 or day 5, with day 0 defined as the day on which non-adherent cells are removed from the culture vessel, or between 84 hours and 132 hours after the removal of the non-adherent cells.

### Embodiment 8: (4) Pulsing Step: Optimal Pulsing Dose

In present Embodiment 8, the manufacturing process is carried out in accordance with the production method described in Embodiment 5. The difference from Embodiment 5 lies in the concentration of α-Galactosylceramide used for pulsing, which is set to 300 ng/ml to 1000 ng/mL. In the present Embodiment 8, α-Galactosylceramide was pulsed 120 hours after the start of culture, which corresponds to the timing when monocytes collected from the beneficiary were placed into the culture vessel.

The dendritic cell preparation produced by the production method of the present invention is expected to activate various components of the immune system by stimulating Vα24-NKT cells, thereby inducing their activation and subsequent production of IFN-γ. For evaluating the efficacy of the preparation, the non-adherent cells (such as lymphocytes (T cells, B cells, NK cells, and NKT cells) or the like) that were removed in step (2) of the production method, namely the non-adherent cell removal step, and separately cultured and preserved, are added to the completed preparation, followed by co-culture. The correlation between the concentration of o-Galactosylceramide used for pulsing and the amount (concentration) of IFN-γ produced by cultured NKT cells among the non-adherent cells after co-culture is examined and confirmed as the effect. The range of pulsing doses described above was determined in that way.

In the present embodiment, time is recorded in hours, with the starting point set as the time when monocytes collected from the beneficiary are placed into a culture vessel and the culture is started. After the completion of the preparation, non-adherent cells were added, and co-culture was performed to confirm the efficacy of the preparation. To verify the optimal pulsing concentration, the collected monocytes were divided into four groups to prepare four samples of dendritic cell preparations, each with a different pulsing timing of α-Galactosylceramide. 120 hours after the monocytes collected from the beneficiary were placed into the culture vessel to start the culture, the cells were pulsed with α-Galactosylceramide at the concentration of 0 ng/ml for Condition 1, 100 ng/ml for Condition 2, 300 ng/mL for Condition 3 and 1000 ng/ml for Condition 4.

To confirm the efficacy of the preparation by evaluating the concentration of IFN-γ production, 168 hours after the start of culture, non-adherent cells were added, and co-culture was started. IFN-γ production amount was examined on day 0 (the day of the start of the co-culture), day 2, day 4, day 7, and day 14 for each condition, when day of the start of the co-culture was defined as day 0.

IFN-γ was measured by ELISA using the ELISA MAX Deluxe Set Human IFN-γ from BIOLEGEND.

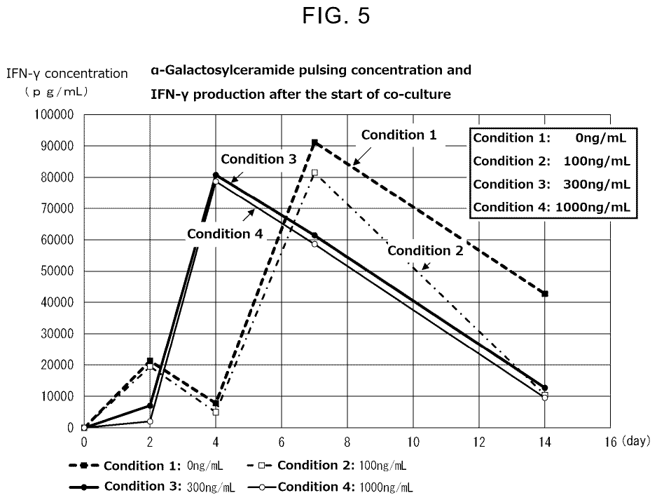

FIG. 5 is a diagram showing the relationship between the concentration of α-Galactosylceramide pulsed at 120 hours after the start of culture, when monocytes collected from the beneficiary were placed into the culture vessel, and the concentration of IFN-γ measured on day 0 (the day of the start of the co-culture), day 2, day 4, day 7, and day 14 after the start of co-culture with the non-adherent cells. The horizontal axis represents the number of days of co-culture, while the vertical axis represents the concentration of IFN-γ (pg/mL). As described above, in Conditions 1 to 4, the concentration of α-Galactosylceramide to be pulsed was varied in four levels, from 0 ng/ml to 1000 ng/mL, for experimentation. As shown in FIG. 5, in Condition 1 (0 ng/ml) and Condition 2 (100 ng/ml), the concentration of IFN-γ production reached its peak on day 7 from the start of co-culture. In Condition 3 (300 ng/ml) and Condition 4 (1000 ng/ml), the production concentration reached its peak on day 4, and the peak values were equivalent to that of Condition 2 (100 ng/ml). In Condition 3 (300 ng/ml) and Condition 4 (1000 ng/ml), the production concentration reached its peak three days earlier than the concentration condition (100 ng/ml) of Embodiment 1 disclosed in the prior art (Patent Document 1, hereinafter the same). This indicates that efficacy was achieved more quickly. As demonstrated, when the concentration of α-Galactosylceramide to be pulsed is below 300 ng/ml, the IFN-γ production peak is delayed, and when the concentration exceeds 1000 ng/ml, the result is the same as that at 1000 ng/ml, meaning that α-Galactosylceramide is wasted. Therefore, the optimal concentration of α-Galactosylceramide was found to be not less than 300 ng/ml and not more than 1000 ng/mL.

Furthermore, in a comparative experiment with the Prior Art (Patent Document 1), described later, when the same concentration of α-Galactosylceramide was pulsed, the present invention, which pulses α-Galactosylceramide to immature dendritic cells, resulted in approximately four times the concentration of IFN-γ production compared to the prior art (Patent Document 1), which pulses α-Galactosylceramide to mature dendritic cells. As a result, the present invention is capable of producing a greater amount of IFN-γ than the prior art, and expected to activate immune system more and exert greater anti-cancer effect.

In the present embodiment, the preparation produced by the production method of the present invention is expected to advance the peak of IFN-γ production in the body of the beneficiary, while maintaining the peak value equivalent to that of other conditions, thereby exerting an anti-cancer effect more quickly. Furthermore, the preparation is expected to achieve a higher concentration of IFN-γ production than the prior art and exert a greater anti-cancer effect.

### <Embodiment 9: Overview> Mainly Corresponding to Claims 9 and 10

Provided is a blood from a beneficiary, to which any one of the dendritic cell preparations produced by the production method according to Embodiments 1 to 8 has been added.

As explained in Embodiment 1 above, when the present preparation is added to the blood of the beneficiary, it stimulates NKT cells, thereby activating the innate immune system (enhancing immunity), followed by stimulating and activating the adaptive immune system. The present preparation has the effect of enhancing immunity in the blood of the beneficiary.

The blood from the beneficiary may be extracted from the body, and the present preparation may be added to process the blood. By reintroducing the processed blood (containing the present preparation) into the beneficiary, the beneficiary's immunity is expected to be enhanced. Alternatively, the present preparation may be directly reintroduced into the beneficiary, where it is added to the blood within the body to enhance immunity. Furthermore, the blood from the beneficiary may be replaced with the semi-blood of the beneficiary, which is a mixture of the beneficiary's blood and transfusable blood that can be safely transfused into the beneficiary.

### Results of Application to the Human Body

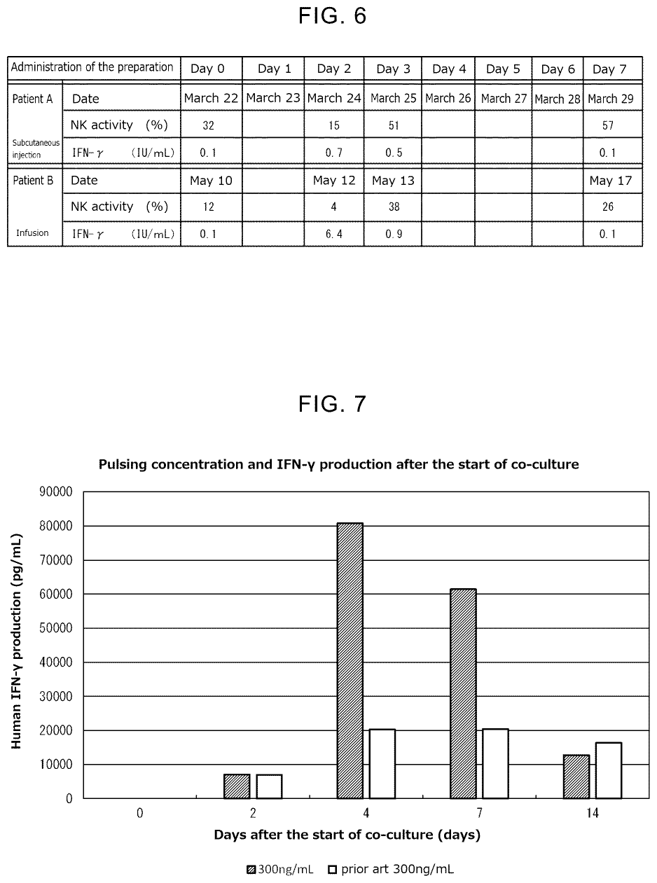

The dendritic cell preparation produced under the conditions of Embodiment 4 (in which culture was started on the day of blood collection, non-adherent cells were removed, GM-CSF and IL-4 were added 24 hours after the start of the culture, and α-Galactosylceramide was pulsed at 300 ng/ml on day 5) was administered to two beneficiaries. The manner how the dendritic cell preparation works in the body is described above. It activates NKT cells, which in turn activate various other immune cells through the production of IFN-γ. Since NK cells are particularly affected, NK activities and IFN-γ levels after the effective administration were examined. The results are shown in FIG. 6. Two beneficiaries were administered, one by subcutaneous injection (upper row of the table) and the other by intravenous infusion (lower row of the table). The NK activity level is a numerical indicator of NK cell activation. NK activity increased from day 3 after administration and was still higher than before administration on day 7. The measured IFN-γ level was 0.1 on the first day (day 0), but peaked on day 2 after administration, and both patients showed a large increase in the level. These results indicate that the present dendritic cell preparation has achieved its objective of activating NKT cells, increasing IFN-γ production quickly, and activating NK cells as well.

### Comparison With the Prior Art: Overview

The prior art does not describe the amount (concentration) of IFN-γ produced in its specification. Therefore, the inventors of the present invention prototyped a cell composition in accordance with the production method of the cell composition described in the prior art, specifically in Patent Document 1, which corresponds to the dendritic cell preparation of the present invention. An experiment was conducted to compare the concentration of IFN-γ production after co-culture with non-adherent cells, including NKT cells.

### Comparison With the Prior Art: Preparation of Comparative Example

To reproduce the prior art, mononuclear cells obtained by apheresis were placed in a culture vessel containing liquid medium in an appropriate position for 2 hours, after which non-adherent cells (lymphocytes (such as T cells, B cells, NK cells, NKT cells)) were separated and cryopreserved. The day when this incubation was performed and the culture of the adherent cells was started was defined as day 0. On the following day (day 1), GM-CSF and IL-4 were added to the culture medium of the adherent cells to induce their differentiation into immature dendritic cells. On day 5, GM-CSF, IL-4, PGE-2, and OK-432 were added to the immature dendritic cells to induce their maturation. On the following day (day 6), the mature dendritic cells were pulsed with α-Galactosylceramide (at a concentration of 300 ng/ml).

### Comparison With the Prior Art: Method for Confirming Effect of the Comparative Example

On day 7, co-culture of the mature dendritic cells with the non-adherent cells collected on the first day is started. The concentration of IFN-γ (pg/mL) produced by NKT cells among the non-adherent cells was measured on day 0 (the day of the start of co-culture), and on day 2, day 4, day 7, and day 14, with day 0 defined as the day on which the co-culture was started.

### Comparison With the Prior Art: Comparison of Effects Between the Comparative Example and the Embodiment of the Present Application

The concentration of IFN-γ produced after co-culture of dendritic cells produced in accordance with the prior art as a comparative example (according to the production method described in Patent Document 1 except that the concentration of pulsed α-Galactosylceramide was set to 300 ng/ml) and the non-adherent cells was compared with the concentration of IFN-γ produced after co-culture of the dendritic cell preparation manufactured under Condition 3 of Embodiment 8 of the present application (in which pulsed the concentration of α-Galactosylceramide was also set to 300 ng/ml) and the non-adherent cells. To verify the effect of the comparative example and the preparation manufactured under Condition 3 of Embodiment 8 of the present application, dendritic cells and non-adherent cells, including NKT cells, which were derived from the same beneficiary, were used, while ensuring that the difference in cell numbers was controlled within a range that would not result in a statistically significant difference.

### Comparison With the Prior Art: Comparison of Effects Between the Comparative Example and the Embodiment of the Present Application: Concentration of IFN-γ Production

FIG. 7 shows the results of the comparison of concentrations of IFN-γ production. The horizontal axis represents the number of days elapsed after the start of co-culture: day 0 (first day of co-culture), day 2, day 4, day 7, and day 14, and the vertical axis represents the concentration of IFN-γ production (pg/mL). In both the comparative example (prior art) and Condition 3 of Embodiment 8 of the present application, the concentration of IFN-γ production is greater on day 4. In the prior art, the concentration of IFN-γ production on day 7 is slightly higher than on day 4. However, Condition 3 of Embodiment 8 of the present application demonstrated a concentration of IFN-γ production approximately four times higher than that of prior art.

### Comparison With the Prior Art: Comparison of Effects Between the Comparative Example and the Embodiment of the Present Application: Peak of IFN-γ Production

In Patent Document 1, which represents the prior art, the most preferred concentration range of α-Galactosylceramide is described as 80 to 120 ng/ml, and in Embodiment 1 of Patent Document 1, experiments were conducted using a concentration of 100 ng/mL. However, Patent Document 1 does not evaluate the effect on IFN-γ production. In the experiments conducted by the inventors of the present application, Condition 2 of Embodiment 8, in which α-Galactosylceramide was pulsed at the same concentration (100 ng/ml) as in Embodiment 1 of Patent Document 1, exhibited a peak of IFN-γ production on day 7 after co-culture. Therefore, although Patent Document 1 does not describe this, the inventors of the present application have found that by optimizing not only the pulsing timing of α-Galactosylceramide but also the pulsing concentration, it is possible to narrow down the range that achieve efficacy more quickly than the prior art.

### Comparison With the Prior Art: Superior Effects of the Present Invention

Compared to the comparative example (prior art), the present invention enables a significant improvement in the concentration of IFN-γ production by pulsing α-Galactosylceramide into immature dendritic cells, optimizing both the timing and concentration of pulsing, and narrowing down the range that allows for earlier IFN-γ production.

### <Proposed Claims Describing the Treatment Method for U.S. Application>: (Tentative)

The Method for treating a beneficiary who desires one or more of cancer treatment, cancer prevention, and immunity enhancement using the NKT-cell stimulating dendritic cell preparation manufactured by the production method of the present invention can be described as follows.

A treatment method by the preparation produced by the present invention, which comprises
(1) a collection step comprising collecting monocytes or peripheral blood containing monocytes collected by blood apheresis or blood collection from a beneficiary,
(2) an adhesion step comprising placing the collected monocytes into a culture vessel with a liquid medium and statically incubating them to allow some of said monocytes to adhere to the inner surface of the vessel,
(3) a non-adherent cell removal step comprising removing non-adherent cells including monocytes, which are cells other than those adhered to the inner surface of the culture vessel.
(4) a differentiation step comprising adding predetermined factors to the culture vessel to allow the monocytes adhered to the inner surface of the culture vessel to differentiate into immature dendritic cells,
(5) a pulsing step comprising pulsing α-Galactosylceramide into the culture vessel in which said immature dendritic cells are present in a non-adherent state, and
(6) an NKT-stimulating dendritic cell induction step comprising inducing NKT-stimulating dendritic cells from the immature dendritic cells, which are dendritic cells that stimulate NKT cells, thereby producing a dendritic cell preparation comprising dendritic cells that stimulate natural killer T (NKT) cells for one or more of the following purposes: treating cancer, preventing cancer, or enhancing immunity, and
(7) a reintroduction step comprising reintroducing said dendritic cell preparation into the body of the beneficiary,
wherein the preparation is the dendritic cell preparation comprising dendritic cells that stimulate natural killer T (NKT) cells.

### (Tentative)

### <Proposed Limitations for the Above Proposed Claim> (Tentative)

One or more of the following limitations may be applied to the treatment method described above.

(a) A treatment method in which-Galactosylceramide is pulsed within the period of 84 hours to 132 hours after monocytes are placed into a culture vessel containing liquid medium (4 days or 5 days after monocytes were placed, with day 0 defined as the day on which monocytes were placed) in the pulsing step.

(b) The treatment method in which the concentration of α-Galactosylceramide to be pulsed in the pulsing step is 300 to 1000 ng/mL.

(c) The treatment method in which reintroduction of the preparation into the body of the beneficiary is carried out 156 hours or later after the placement of the monocytes into the culture vessel with a liquid medium (7 days or later after monocytes were placed, with day 0 defined as the day on which monocytes were placed) in the reinfusion step.

### Another Embodiment: Cocktail Preparation

The following describes the effects observed upon reintroducing a cocktail preparation of another embodiment into the body of a beneficiary. In the example described in this explanation, the beneficiary has cancer cells in the body. In this alternative embodiment, in addition to the preparation of the present invention, collected monocytes are divided and placed into a culture vessel, and a preparation is manufactured in parallel in a separate vessel, in which WT1 peptide is used as a ligand instead of α-Galactosylceramide, which is pulsed into immature dendritic cells in the present invention. This alternative preparation is configured to be present in the body of the beneficiary simultaneously with the preparation of the present invention.

A preparation using α-Galactosylceramide as a ligand activates innate immune system in the beneficiary, thereby enhancing immunity and indirectly activating acquired immune system. In contrast, a preparation using WT1 peptide as a ligand, which is present in various types of cancer, directly activates the acquired immune system targeting cancer cells.

### Action of Cocktail Preparation

This alternative embodiment utilizes two types of preparations: one manufactured using the production method of the present invention and another manufactured using a similar production method, except that WT1 peptide is pulsed into immature dendritic cells instead of α-Galactosylceramide. Since nonspecific immunotherapy (preparation with α-Galactosylceramide as a ligand) has already been described, the following will primarily explain the effects of the preparation using WT1 peptide as a ligand, which specifically targets the adaptive immune system. In the body of the beneficiary, cancer cells form a barrier around themselves to evade immune function due to immunosuppressive effects.

### Action of a Preparation Pulsed With α-Galactosylceramide: Activation of NKT Cells

A preparation pulsed with α-Galactosylceramide activates NKT cells. Activated NKT cells exert various effects on immunity.

### Action of Activated NKT Cells: Adjuvant Action

The activated NKT cells produce cytokines (IFN-γ), thereby activating NK cells, macrophages, and killer T cells, enhancing their ability to attack cancer. Additionally, various immune cells are activated. Killer T cells and helper T cells are activated as a result of WT1 peptide, a specific immune activator, being used as a ligand and pulsed into dendritic cells. The simultaneous activation of these activated NK cells and activated B cells, which receive the beneficial effects of the nonspecific immune activation pathway, results in a synergistic effect that exceeds a simple additive effect. This is one of the distinguishing features of this alternative embodiment.

### Action of Activated NKT Cells: Induction of Apoptosis in Cancer Cells

Activated NKT cells produce a type of serine protease that degrades the DNA of cancer cells, inducing apoptosis in the cancer cells.

### Action of Activated Macrophages: Phagocytosis of Cancer Cells by Macrophages

Activated macrophages phagocytose cancer cells and dead cancer cells.

### Action of Proliferated and Activated Macrophages: Antigen-Presenting Action of Macrophages

Activated NKT cells induce the proliferation and activation of macrophages. The proliferated and activated macrophages fragment the cancer cells they have engulfed through phagocytosis and present the fragments on their cell surface. In other words, the macrophages exhibit an antigen-presenting function by presenting antigens from cancer cells. The macrophages subsequently present these antigens to helper T cells.

### Cancer Antigen-Presenting Function of Dendritic Cells: Cancer Antigen Presentation by Dendritic Cells

Immature dendritic cells incorporate cancer antigens, undergo maturation, and differentiate into mature dendritic cells, which then present cancer antigens to helper T cells and killer T cells. This function is an anti-cancer function naturally occurring within the body; however, due to the effect of activated NKT cells, an enhanced anti-cancer effect can be achieved beyond normal levels. Since these cancer antigens originate from the patient's own cancer cells and are presented by dendritic cells and macrophages that have phagocytosed said cancer cells, they are expected to be more effective than preparations using artificial antigens as ligands. This function is also one of the factors contributing to the synergistic effect of the cocktail preparation of the present invention.

### Action of the Preparation Pulsed With WT1 Peptide: Cancer Antigen-Presenting Function

The preparation pulsed with WT1 peptide presents WT1 peptide, a cancer antigen, to helper T cells and killer T cells, thereby activating them. As a result, killer T cells recognize cancer cells as targets for attack, while helper T cells activate killer T cells and further produce cytokines, which in turn activate NK cells and promote B cells to produce antibodies, as described below.

### Action of Activated Helper T Cells: Instruction for Attacking Cancer Cells

Activated helper T cells produce cytokines (IFN-γ) and, based on the information contained in these cytokines, instruct killer T cells to attack cancer cells. Additionally, due to the effects of cytokines, NK cells, B cells, and macrophages are activated, thereby enhancing the anti-cancer attack function.

### Action of Activated Helper T Cells: Instruction for Antibody Production Against Cancer Antigens (e.g., WT1)

Activated helper T cells produce cytokines and, based on the information contained in these cytokines, instruct B cells to produce antibodies against cancer antigens (e.g., WT1).

### Action of Activated Killer T Cells: Cancer Cell Attack Action by Killer T Cells

In this action, which is the main force directly attacking cancer cells, activated killer T cells recognize and bind to WT1 expressed on cancer cells, perforating the cell membrane and inducing necrosis in the cancer cells. Additionally, activated killer T cells produce substances such as TNF-β (tumor necrosis factor, a type of cytokine) and induce apoptosis of cancer cells. Thus, the direct attack of killer T cells on cancer cells is a powerful anti-cancer function that attacks cancer cells from both outside and inside.

### Action of Activated Killer T Cells: Neovascular Destruction Function

Since blood vessels newly formed by cancer cells express the WT1 gene, killer T cells that have been presented with the cancer antigen (WT1) attack not only cancer cells but also newly formed blood vessels. As a result, newly formed blood vessels are destroyed, the supply of nutrients to cancer cells is reduced, and the proliferation of cancer cells is suppressed. This action is also one of the factors contributing to the synergistic effect of the cocktail preparation in this embodiment.

### Action of Activated NK Cells: Cancer Cell Attack Action by NK Cells

Activated NK cells, which are stimulated by IFN-γ produced not only by activated NKT cells but also by helper T cells, exhibit cytotoxic response against cancer cells and attack them.

### Action of Activated B Cells: Cancer Antibody Production

Activated B cells produce antibodies against cancer antigens (e.g., WT1) and attack cancer cells. These antibodies function by binding to cancer cells, thereby suppressing cancer cell proliferation and serving as a marker for other immune cells (e.g., NK cells and killer T cells) to facilitate their attack on cancer cells.

As described above, by reintroducing both the preparation using α-Galactosylceramide as a ligand and the preparation using WT1 peptide as a ligand into the body of the beneficiary so that they coexist simultaneously, it is possible to activate the acquired immune system targeting cancer cells more quickly than when using the preparation of the present invention (the preparation using α-Galactosylceramide as a ligand) alone. Furthermore, by directly activating both the innate immune system and the acquired immune system, a synergistic effect beyond a simple additive effect can be obtained. Therefore, for the purpose of treating cancer, this approach is expected to produce faster and greater effects than the present preparation alone.

### Effects

According to the present method for producing a dendritic cell preparation that stimulates NKT cells, which is for reintroducing into the body of a beneficiary from whom the monocytes were collected, the amount of IFN-γ production by NKT cells can be greater than in the prior art (Patent Document 1) and the range of α-Galactosylceramide concentration that allows for earlier IFN-γ production can be narrowed down, thereby achieving superior effects in that a dendritic cell preparation can be generated, which is expected to exhibit an enhanced therapeutic effect by allowing for a greater expected effect through both the innate immune system and the adaptive immune system pathways in cancer treatment and other applications. In addition, the present dendritic cell preparation above also exhibits the effect of enhancing immunity in healthy people. By manufacturing the preparation from the beneficiary's autologous monocytes, safe preparation can be obtained without concerns of rejection.

1. A method for producing a dendritic cell preparation comprising dendritic cells that stimulate natural killer T (NKT) cells for one or more of the following purposes: treating cancer, preventing cancer, or enhancing immunity which comprises
(1) an adhesion step comprising placing monocytes collected from a beneficiary (monocytes or peripheral blood containing monocytes collected by blood apheresis or blood collection) into a culture vessel with a liquid medium and statically incubating them to allow some of said monocytes to adhere to the inner surface of the vessel,
(2) a non-adherent cell removal step comprising removing non-adherent cells including monocytes, which are cells other than those adhered to the inner surface of the culture vessel,
(3) a differentiation step comprising adding predetermined factors to the culture vessel to allow the monocytes adhered to the inner surface of the culture vessel to differentiate into immature dendritic cells,
(4) a pulsing step comprising pulsing α-galactosylceramide into the culture vessel in which said immature dendritic cells are present in a dominant state, and
(5) an NKT-stimulating dendritic cell induction step comprising inducing NKT-stimulating dendritic cells from the immature dendritic cells to NKT-stimulated dendritic cells, which are dendritic cells that stimulate NKT cells,
wherein the dendritic cell preparation is a NKT cell-stimulating dendritic cell preparation for reintroducing into the body of the beneficiary from whom the monocytes were collected, to stimulate NKT cells.

2. The method for producing a dendritic cell preparation according to claim 1, wherein the pulsing step (4) is carried out on day 3 or day 4, wherein day 0 is defined as the day on which the predetermined factors are added in the differentiation step (3).

3. The method for producing a dendritic cell preparation according to claim 1, wherein the pulsing step (4) is carried out within a period of 60 to 108 hours after the predetermined factors have been added in the differentiation step (3).

4. The method for producing a dendritic cell preparation according to claim 1, wherein the pulsing step (4) is carried out on day 4 or day 5, wherein day 0 is defined as the day on which the monocytes collected from the beneficiary are placed into the culture vessel in the adhesion step (1).

5. The method for producing a dendritic cell preparation according to claim 1, wherein the pulsing step (4) is carried out within a period of 84 to 132 hours after the monocytes collected from the beneficiary have been placed into the culture vessel in the adhesion step (1).

6. The method for producing a dendritic cell preparation according to claim 1, wherein the pulsing step (4) is carried out on day 4 or day 5, wherein day 0 is defined as the day on which the non-adherent cells including monocytes, which are cells other than those adhered to the inner surface of the culture vessel, are removed in the non-adherent cell removal step (2).

7. The method for producing a dendritic cell preparation according to claim 1, wherein the pulsing step (4) is carried out within a period of 84 to 132 hours after the non-adherent cells including monocytes, which are cells other than those adhered to the inner surface of the culture vessel, are removed in the non-adherent cell removal step (2).

8. The method for producing a dendritic cell preparation according to claim 1, wherein the concentration of α-galactosylceramide to be pulsed in the pulsing step (4) is from 300 to 1000 ng/mL.

9. Blood from a beneficiary, to which the dendritic cell preparation produced by the production method according to claim 1 has been added.

10. Blood from a beneficiary, to which the dendritic cell preparation produced by the production method according to claim 8 has been added.
